(19) United States
(12) Patent Application Publication
(10) Pub. No.: US 2024/0002428 A1
(43) Pub. Date: Jan. 4, 2024

(54) STORAGE STABLE CAGED HAPTENS

(71) Applicant: VENTANA MEDICAL SYSTEMS, INC., Tucson, AZ (US)

(72) Inventors: Yuri Belosludtsev, Tucson, AZ (US); Brian D. Kelly, Tucson, AZ (US); Nathan W. Polaske, Oracle, AZ (US)

(21) Appl. No.: 18/220,207

(22) Filed: Jul. 10, 2023

Related U.S. Application Data
(63) Continuation of application No. PCT/EP2022/050602, filed on Jan. 13, 2022.
(60) Provisional application No. 63/137,805, filed on Jan. 15, 2021.

(51) Int. Cl. C07J 19/00; G01N 33/58; A61K 47/68

(52) U.S. Cl. CPC C07J 19/00; G01N 33/58; A61K 47/6803

(57) ABSTRACT

Disclosed herein are caged haptens and caged hapten-antibody conjugates useful for facilitating the detection of targets located proximally to each other in a sample.

### CROSS-REFERENCE TO RELATED APPLICATIONS

The present application is a continuation of International Application No. PCT/EP2022/050602 filed on Jan. 13, 2022, which application claims the benefit of the filing date of U.S. Provisional Patent Application No. 63/137,805 filed on Jan. 15, 2021, the disclosure of which is hereby incorporated by reference herein in its entirety.

### FIELD OF THE INVENTION

Disclosed embodiments concern detecting targets in a sample, including targets located proximally in a sample. Disclosed embodiments also provide for a proximity assay for detecting protein dimers in formalin-fixed, paraffin embedded tissue using caged haptens or caged hapten conjugates.

### STATEMENT OF INDUSTRIAL APPLICABILITY

The present disclosure has industrial applicability in the fields of chemistry and diagnostics.

### BACKGROUND OF THE DISCLOSURE

Immunohistochemistry (IHC) refers to the processes of detecting, localizing, and/or quantifying antigens, such as a protein, in a biological sample using antibodies specific to the particular antigens. IHC provides the substantial advantage of identifying exactly where a particular protein is located within the tissue sample. It is also an effective way to examine the tissues themselves. In situ hybridization (ISH) refers to the process of detecting, localizing, and quantifying nucleic acids. Both IHC and ISH can be performed on various biological samples, such as tissue (e.g., fresh frozen, formalin fixed, paraffin embedded) and cytological samples. Recognition of the targets can be detected using various labels (e.g., chromogenic, fluorescent, luminescent, radiometric), irrespective of whether the target is a nucleic acid or an antigen. To robustly detect, locate, and quantify targets in a clinical setting, amplification of the recognition event is desirable as the ability to confidently detect cellular markers of low abundance becomes increasingly important for diagnostic purposes. For example, depositing at the marker's site hundreds or thousands of label molecules in response to a single antigen detection event enhances, through amplification, the ability to detect that recognition event.

Networks of protein-protein interactions are the hallmarks of biological systems. Protein-protein interactions form signal pathways that regulate all aspects of cellular functions in normal and cancerous cells. Methods have been developed for detecting protein-protein interactions, such as transient receptor tyrosine kinase dimerization and complex formation after extracellular growth factor activation; however, these methods are not particularly designed to be used on formalin fixed paraffin embedded (FFPE) tissues.

The ability to interrogate for presence and distribution of specific intermolecular interactions for biomarkers known to be important determinants in cancer biology is of high interest in the context of new diagnostic capabilities and for determining therapeutic effect in the context of pharmaceutical development. The ability to probe and document distributions of molecular interactions on frozen and paraffin embedded tissue has remained inaccessible; alternative technologies to approach this question have been proposed, although the solutions have not proven to be effective and reliable under practical use.

A proximity ligation assay has been developed by Olink AB. This is the only known commercial product for in situ detection of protein-protein interactions on formalin fixed paraffin embedded tissue. Proximity ligation assay technology uses DNA ligases to generate a padlock circular DNA template, as well as Phi29 DNA polymerase for rolling circle amplification. These enzymes are expensive. Moreover, these enzymes are not amenable for use with automated systems and methods. For these reasons, proximity ligation assays are not considered generally useful for commercial applications.

### BRIEF SUMMARY OF THE DISCLOSURE

A first aspect of the present disclosure is a caged hapten having any one of Formulas (IA) and (IB):

R²—R¹—O-[DIG]-[Phosphoryl] (IA),

R²—R¹—O-[DIG]-PO₄H₂ (IB),

wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
[DIG] is digoxigenin;
[Phosphoryl] is represented by the formula:

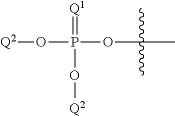

Q¹ is O or S; and
Q² is H, —CH₃, or —CH₂CH₃;
where the group [Phosphoryl] or the group —PO₄H₂ may be attached to any position of [DIG].

In some embodiments, Q¹ is S.

In some embodiments, Q¹ is O, and at least one Q² is H. In some embodiments, R² is selected from an amine-reactive group, a thiol-reactive group, and a carbonyl-reactive group. In some embodiments, R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

In some embodiments, both Q² groups are H. In some embodiments, R² is selected from an amine-reactive group, a thiol-reactive group, and a carbonyl-reactive group. In some embodiments, R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group. In some embodiments, R¹ has the structure depicted in Formula (IIIA):

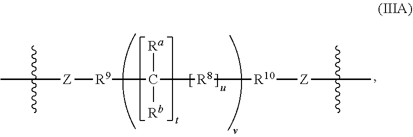

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

In some embodiments, at least one of R^{a} or R^{b} is H. In some embodiments, R⁸ is O. In some embodiments, R⁸ is a bond. In some embodiments, at least one of R^{a} or R^{b} is H. In some embodiments, both R^{a} and R^{b} are H. In some embodiments, Z is a bond or —CH₂—.

In some embodiments, R¹ has the structure depicted in Formula (IIIC):

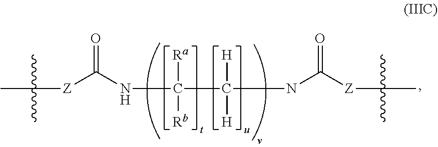

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

In some embodiments, at least one of R^{a} or R^{b} is H. In some embodiments, Z is a bond or —CH₂—. In some embodiments, both Q² groups are H. In some embodiments, R² is selected from an amine-reactive group, a thiol-reactive group, and a carbonyl-reactive group.

In some embodiments, R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group. In some embodiments, wherein Q¹ is O.

A second aspect of the present disclosure is a caged hapten having Formula (IIID):

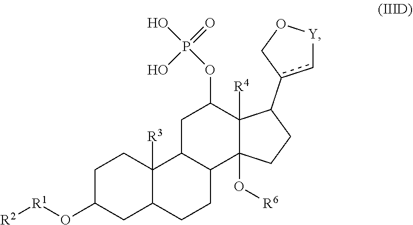

wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
R⁶ is H or a linear or branched or substituted or unsubstituted C₁-C₆ alkyl group;
m, n, and o are each independently 0 or an integer ranging from 1 to 4; and
Y is —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

In some embodiments, R¹ has the structure depicted in Formula (IIIC):

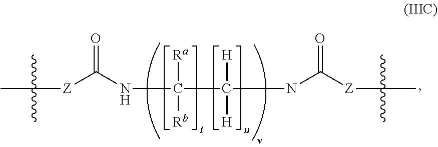

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

In some embodiments, at least one of R^{a} or R^{b} is H. In some embodiments, Z is a bond or —CH₂—. In some embodiments, R² is selected from an amine-reactive group, a thiol-reactive group, and a carbonyl-reactive group. In some embodiments, R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group. In some embodiments, R² is selected from an amine-reactive group, a thiol-reactive group, and a carbonyl-reactive group. In some embodiments, R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

In some embodiments, R¹ has the structure depicted in Formula (IIIA):

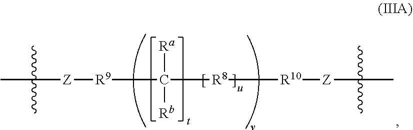

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

In some embodiments, at least one of R^{a} or R^{b} is H. In some embodiments, R⁸ is 0. In some embodiments, R⁸ is a bond. In some embodiments, at least one of R^{a} or R^{b} is H. In some embodiments, both R^{a} and R^{b} are H. In some embodiments, Z is a bond or —CH₂—. In some embodiments, at least one of R³, R⁴, or R⁶ is —CH₃. In some embodiments, at least one of R³ and R⁴ is —CH₃. In some embodiments, R⁶ is H. In some embodiments, R² is H. In some embodiments, Y is —C(O)—. In some embodiments, R² is H and Y is —C(O)—. In some embodiments, R¹ has the structure depicted in Formula (IIIA):

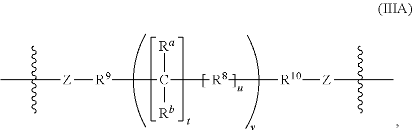

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

A third aspect of the present disclosure is a conjugate comprising (i) any one of the caged haptens described in the first and second aspects above, and (ii) and a primary antibody. In some embodiments, the caged hapten is indirectly coupled to the primary antibody. In some embodiments, the primary antibody is an intact primary antibody.

A fourth aspect of the present disclosure is a conjugate comprising (i) any one of the caged haptens described in the first and second aspects above, and (ii) and a secondary antibody. In some embodiments, the caged hapten is indirectly coupled to the secondary antibody. In some embodiments, the secondary antibody is an intact secondary antibody.

A fifth aspect of the present disclosure is a conjugate having any one of Formulas (IVA) and (IVB):

[Specific Binding Entity]-W¹—W²—R¹—O-[DIG]-[Phosphoryl] (IVA),

[Specific Binding Entity]-W¹—W²—R¹—O-[DIG]-PO₄H₂ (IVB),

wherein
W¹ is a bond, or a group comprising a branched or unbranched, substituted or unsubstituted, saturated or unsaturated aliphatic group having between 1 and 10 carbon atoms, and optionally including one or more heteroatoms selected from the group consisting of O, N, or S;
W² is derived from a reactive functional group;
[DIG] is digoxigenin;
[Phosphoryl] is represented by the formula:

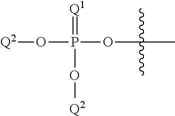

Q¹ is O or S;
Q² is H, —CH₃, or —CH₂CH₃; and
[Specific Binding Entity] is a specific binding entity;
where the group [Phosphoryl] or the group —PO₄H₂ may be attached to any position of [DIG].

In some embodiments, the [Specific Binding Entity] is an antibody. In some embodiments, the [Specific Binding Entity] is a monoclonal antibody. In some embodiments, the [Specific Binding Entity] is a primary antibody. In some embodiments, the [Specific Binding Entity] is a secondary antibody.

In some embodiments, the conjugate has Formula (IVA), and wherein the [Specific Binding Entity] is a monoclonal antibody. In some embodiments, the conjugate has Formula (IVB), and wherein the [Specific Binding Entity] is a monoclonal antibody.

In some embodiments, Q¹ is O, and at least one Q² is H. In some embodiments, W² is derived from an amine-reactive group, a thiol-reactive group, and a carbonyl-reactive group. In some embodiments, W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group. In some embodiments, both Q² groups are H. In some embodiments, W² is derived from an amine-reactive group, a thiol-reactive group, and a carbonyl-reactive group. In some embodiments, W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group.

In some embodiments, R¹ has the structure depicted in Formula (IIIA):

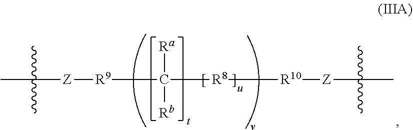

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c}(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8. In some embodiments, v ranges from 1-4.

In some embodiments, R^{a} and R^{b} are each independently H, a C₁-C₂ alkyl group, F, Cl, or —N(R^{c})(R^{d}). In some embodiments, R^{a} and R^{b} are each independently H or a C₁-C₂ alkyl group.

In some embodiments, at least one of R^{a} or R^{b} is H. In some embodiments, R⁸ is O. In some embodiments, R⁸ is a bond. In some embodiments, at least one of R^{a} or R^{b} is H. In some embodiments, both R^{a} and R^{b} are H. In some embodiments, Z is a bond or —CH₂—.

In some embodiments, R¹ has the structure depicted in Formula (IIIC):

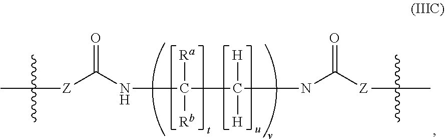

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1-4.

In some embodiments, R^{a} and R^{b} are each independently H, a C₁-C₂ alkyl group, F, Cl, or —N(R^{c})(R^{d}). In some embodiments, R^{a} and R^{b} are each independently H or a C₁-C₂ alkyl group.

In some embodiments, at least one of R^{a} or R^{b} is H. In some embodiments, Z is a bond or —CH₂—. In some embodiments, both Q² groups are H. In some embodiments, W² is derived from an amine-reactive group, a thiol-reactive group, and a carbonyl-reactive group. In some embodiments, W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group. In some embodiments, Q¹ is O.

A sixth aspect of the present disclosure is a method of analyzing a sample to determine whether a first target is proximal to a second target, the method comprising: contacting the sample with an unmasking enzyme-antibody conjugate to form a target-unmasking enzyme-antibody conjugate complex; contacting the sample with any one of the caged hapten-antibody conjugates described above with regard to the third, fourth, and fifth aspects of the present disclosure to form a target-caged hapten-antibody conjugate complex; unmasking the caged hapten of the target-caged hapten-antibody conjugate complex to form a target-unmasked hapten-antibody conjugate complex; contacting the sample with first detection reagents to label the first target-unmasked hapten-antibody conjugate complex or the first target; and detecting the labeled first target-unmasked hapten-antibody conjugate complex or labeled first target. In some embodiments, the caged hapten-antibody conjugate includes a monoclonal antibody.

In some embodiments, the first detection reagents comprise (i) a secondary antibody specific to the unmasked hapten of the target-unmasked hapten-antibody complex, the secondary antibody conjugated to a first enzyme such that the secondary antibody labels the target-unmasked hapten-antibody complex with the first enzyme; and (ii) a first substrate for the first enzyme.

In some embodiments, in the first substrate is a chromogenic substrate or a fluorescent substrate.

In some embodiments, the first detection reagents include amplification components to label the unmasked enzyme of the target-unmasked hapten-antibody conjugate complex with a plurality of first reporter moieties.

In some embodiments, the plurality of first reporter moieties are haptens.

In some embodiments, the first detection reagents further comprise secondary antibodies specific to the plurality of first reporter moieties, each secondary antibody conjugated to a second reporter moiety.

A seventh aspect of the present disclosure is a method for analyzing a sample to determine whether a first target is proximal to a second target, the method comprising: contacting the sample with an unmasking enzyme-antibody conjugate to form a target-unmasking enzyme-antibody conjugate complex; contacting the sample with any one of the caged hapten-antibody conjugates described above with regard to the third, fourth, and fifth aspects of the present disclosure to form a target-caged hapten-antibody conjugate complex; unmasking the caged hapten of the target-caged hapten-antibody conjugate complex to form a target-unmasked hapten-antibody conjugate complex; performing a signal amplification step to label the target-unmasked hapten-antibody conjugate complex with a plurality of reporter moieties; and detecting the plurality of reporter moieties. In some embodiments, the caged hapten-antibody conjugate includes a monoclonal antibody.

In some embodiments, the plurality of reporter moieties are haptens; and wherein the method further comprises introducing secondary antibodies specific to the plurality of first reporter moieties, each secondary antibody conjugated to a second reporter moiety. In some embodiments, the second reporter moiety is an amplification enzyme and wherein the method further comprises introducing a chromogenic substrate or a fluorescent substrate for the amplification enzyme. In some embodiments, the method further comprises detecting a total amount of target in the sample.

An eighth aspect of the present disclosure is a method for analyzing a sample to determine whether a first target is proximal to a second target, the method comprising: contacting the sample with a first detection probe, the first detection probe comprising one of the caged hapten-antibody conjugate any one of the caged hapten-antibody conjugates described above with regard to the third, fourth, and fifth aspects of the present disclosure or an unmasking enzyme-antibody conjugate; contacting the sample with a second detection probe, the second detection probe comprising the other of the caged hapten-antibody conjugate of any one of the caged hapten-antibody conjugates described above with regard to the third, fourth, and fifth aspects of the present disclosure to or the unmasking enzyme-antibody conjugate; contacting the sample with at least first detection reagents to label a formed unmasked hapten-antibody conjugate target complex; and detecting signals from the labeled unmasked hapten-antibody conjugate target complex.

In some embodiments, the method further comprises the step of detecting a total amount of target within the sample. In some embodiments, the first detection reagents include amplification components to label the unmasking enzyme of the first target-unmasked hapten-antibody conjugate complex with a plurality of first reporter moieties. In some embodiments, the plurality of first reporter moieties are haptens. In some embodiments, the first detection reagents further comprise secondary antibodies specific to the plurality of first reporter moieties, each secondary antibody conjugated to a second reporter moiety. In some embodiments, the second reporter moiety is selected from the group consisting of an amplification enzyme or a fluorophore. In some embodiments, the second reporter moiety is an amplification enzyme and wherein the first detection reagents further comprise a first chromogenic substrate or fluorescent substrate for the amplification enzyme. In some embodiments, the method further comprises a decaging step.

### BRIEF DESCRIPTION OF THE FIGURES

The patent or application file contains at least one drawing executed in color. Copies of this patent or patent application publication with color drawings will be provided to the Office upon request and the payment of the necessary fee.

FIG. 1 illustrates the carbon numbering of digoxigenin (“DIG”). In this example, a phosphate group is coupled to the 12-position of digoxigenin.

FIG. 2 is a schematic illustrating the interaction between an unmasking enzyme-antibody conjugate comprising an alkaline phosphatase (bound to Target 2) and a caged hapten-antibody conjugate (bound to Target 1), where the unmasking enzyme of the unmasking enzyme-antibody conjugate reacts with an enzyme substrate portion of the caged hapten-antibody conjugate (by virtue of the proximity of Target 1 and Target 2 to each other) to provide the respective unmasked hapten, which may be detected.

FIG. 3 is a schematic illustrating an unmasking enzyme-antibody conjugate (bound to Target 2) and a caged hapten-antibody conjugate (bound to Target 1) where the two targets are not in close proximity to each other, such that the unmasking enzyme of the unmasking enzyme-antibody conjugate does not interact with an enzyme substrate portion of the caged hapten-antibody conjugated, and thus the caged hapten remains masked and unable to be detected.

FIG. 4 provides a flowchart illustrating the steps of detecting protein dimers and/or total protein in a sample.

FIG. 5 is a schematic illustrating an embodiment of an IHC staining protocol where a single antigen is detected with a secondary antibody labeled with caged DIG.

FIG. 6 is a schematic illustrating the uncaging (or unmasking) of a caged DIG, namely a phosphorylated DIG, to provide the native DIG hapten.

FIG. 7 is a schematic illustrating multiplex detection of both proteins (Target 1 and Target 2) in close proximity and total protein (Target 2).

FIG. 8 illustrates the coupling of an antibody to a caged hapten, namely a phosphorylated DIG.

FIG. 9 illustrates the hydrolysis of the caging groups on caged nitrophenyl (NP) and caged DIG to form the native haptens (NP and DIG).

FIG. 10 illustrates an experiment monitoring the amount of caged hapten hydrolyzed (non-enzymatically cleaved by water) to the native hapten expressed as a percentage of the original material for two different caged NP molecules and caged DIG.

FIG. 11A depicts a representative image of a positive proximity assay for E-Cadherin & Beta-Catenin on tonsil tissue using caged NP.

FIG. 11B depicts a representative image of a positive proximity assay for E-Cadherin & Beta-Catenin on tonsil tissue using a caged DIG.

### DETAILED DESCRIPTION

Disclosed herein are caged haptens and their method of synthesis. Also disclosed herein are conjugates comprising a caged hapten. As will be described in more detail herein, the caged hapten conjugates may be used to detect proximal antigens in tissue samples. These and other embodiments are described herein.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “alkaline phosphatase” (AP) refers to an enzyme that removes (by hydrolysis) and transfers phosphate group organic esters by breaking the phosphate-oxygen bond, and temporarily forming an intermediate enzyme-substrate bond. For example, AP hydrolyzes naphthol phosphate esters (a substrate) to phenolic compounds and phosphates. The phenols couple to colorless diazonium salts (chromogen) to produce insoluble, colored azo dyes.

As used herein, the terms “alkyl,” “aromatic,” “heteroalkyl,” “cycloalkyl,” etc. include both substituted and unsubstituted forms of the indicated radical. In that regard, whenever a group or moiety is described as being “substituted” or “optionally substituted” (or “optionally having” or “optionally comprising”) that group may be unsubstituted or substituted with one or more of the indicated substituents. Likewise, when a group is described as being “substituted or unsubstituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₃)).

As used herein, the term “antibody” (Ab) refers to a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH₁, CH₂ and CH₃. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

An immunoglobulin may derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins and includes a monovalent and a divalent fragment or portion, and a single chain antibody.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic, or other techniques known to those skilled in the art.

As used herein, the phrase “antibody conjugates,” refers to those antibodies conjugated (either directly or indirectly) to one or more labels, where the antibody conjugate is specific to a particular target and where the label is capable of being detected (directly or indirectly), such as with a secondary antibody (an anti-label antibody). For example, an antibody conjugate may be coupled to a hapten such as through a polymeric linker and/or spacer, and the antibody conjugate, by means of the hapten, may be indirectly detected. As an alternative example, an antibody conjugate may be coupled to a chromogen, such as through a polymeric linker and/or spacer, and the antibody conjugate may be detected directly. Antibody conjugates are described further in US Publication No. 2014/0147906 and U.S. Pat. Nos. 8,658,389; 8,686,122; 8,618,265; 8,846,320; and 8,445,191. By way of a further example, the term “antibody conjugates” includes those antibodies conjugated to an enzyme, e.g., HRP or AP. In some embodiments, the antibody conjugates include a monoclonal antibody. In other embodiments, the antibody conjugates include a polyclonal antibody.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids, and proteins.

As used herein, the term “aryl” means an aromatic carbocyclic radical or a substituted carbocyclic radical containing preferably from 6 to 10 carbon atoms, such as phenyl or naphtyl or phenyl or naphtyl, optionally substituted by at least one of the substituents selected in the group constituted by alkyl, alkenyl, alkynyl, aryl, aralkyl, hydroxy, alkoxy, aryloxy, aralkoxy, carboxy, aroyl, halo, nitro, trihalomethyl, cyano, alkoxycarbonyl, aryloxycarbonyl, aralkoxycarbonyl, acylamino, aroylamino, carbamoyl, alkylcarbamoyl, dialkylcarbamoyl, alkylthio, arylthio, alkylene or —NYY′ where Y and Y′ are independently hydrogen, alkyl, aryl, or aralkyl.

As used herein, the term a “biological sample” can be any solid or fluid sample obtained from, excreted by, or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease). A biological sample can also be a sample obtained from any organ or tissue (including a biopsy or autopsy specimen, such as a tumor biopsy) or can include a cell (whether a primary cell or cultured cell) or medium conditioned by any cell, tissue, or organ. In some examples, a biological sample is a nuclear extract. In certain examples, a sample is a quality control sample, such as one of the disclosed cell pellet section samples. In other examples, a sample is a test sample. Samples can be prepared using any method known in the art by of one of ordinary skill. The samples can be obtained from a subject for routine screening or from a subject that is suspected of having a disorder, such as a genetic abnormality, infection, or a neoplasia. The described embodiments of the disclosed method can also be applied to samples that do not have genetic abnormalities, diseases, disorders, etc., referred to as “normal” samples. Samples can include multiple targets that can be specifically bound by one or more detection probes.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl or aryl group, or the total number of carbon atoms and heteroatoms in a heteroalkyl, heterocyclyl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃, CH₃CH₂—, CH₃CH₂CH₂—, (CH₃)₂CH, CH₃CH₂CH₂CH₂, CH₃CH₂CH(CH₃) and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, the term “conjugate” refers to two or more molecules or moieties (including macromolecules or supra-molecular molecules) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules moieties. In other embodiments, a conjugate includes one or more specific-binding molecules (such as antibodies) covalently linked to one or more detectable labels (such as a fluorophore, a luminophore, fluorescent nanoparticles, haptens, enzymes and combinations thereof).

As used herein, the term “contacting” is used herein interchangeably with the following: combined with, added to, mixed with, passed over, incubated with, etc.

As used herein, the terms “couple” or “coupling” refers to the joining, bonding (e.g., covalent bonding), or linking of one molecule or atom to another molecule or atom.

As used herein, “cycloalkyl” of like terms (e.g., a cyclic alkyl group) refer to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, the term “chromophore” refers to a molecule or a part of a molecule (e.g., a chromogenic substrate) responsible for its color. Color arises when a molecule absorbs certain wavelengths of visible light and transmits or reflects others. A molecule having an energy difference between two different molecular orbitals falling within the range of the visible spectrum may absorb visible light and thus be aptly characterized as a chromophore. Visible light incident on a chromophore may be absorbed thus exciting an electron from a ground state molecular orbital into an excited state molecular orbital.

As used herein, the term “conjugate” refers to two or more molecules or moieties (including macromolecules or supra-molecular molecules) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules moieties.

As used herein, the term “detectable moiety” refers to a molecule or material that can produce a detectable (such as visually, electronically, or otherwise) signal that indicates the presence (i.e., qualitative analysis) and/or concentration (i.e., quantitative analysis) of the label in a sample.

As used herein, the term “epitopes” refers to an antigenic determinant, such as continuous or non-continuous peptide sequences on a molecule that are antigenic, i.e., that elicit a specific immune response. An antibody binds to a particular antigenic epitope.

As used herein, the terms “halogen atom” or “halogen” mean any one of the radio-stable atoms of column 7 of the Periodic Table of the Elements, such as, fluorine, chlorine, bromine, and iodine.

As used herein, the term “hapten” refers to small molecules that can combine specifically with an antibody, but typically are substantially incapable of being immunogenic except in combination with a carrier molecule. In some embodiments, haptens include, but are not limited to, pyrazoles (e.g., nitropyrazoles); nitropheny compounds; benzofurazans; triterpenes; ureas (e.g., phenyl ureas); thioureas (e.g., phenyl thioureas); rotenone and rotenone derivatives; oxazole (e.g., oxazole sulfonamides); thiazoles (e.g., thiazole sulfonamides); coumarin derivatives; and cyclolignans. Additional non-limiting examples of haptens include thiazoles; nitroaryls; benzofurans; triperpenes; and cyclolignans. Specific examples of haptens include di-nitrophenyl, biotin, digoxigenin, and fluorescein, and any derivatives or analogs thereof. Other haptens are described in U.S. Pat. Nos. 8,846,320; 8,618,265; 7,695,929; 8,481,270; and 9,017,954, the disclosures of which are incorporated herein by reference in their entirety. The haptens themselves may be suitable for direct detection, i.e., they may give off a suitable signal for detection.

As used herein, the term “heteroatom” is meant to include boron (B), oxygen (O), nitrogen (N), sulfur (S), phosphorus (P), and silicon (Si). In some embodiments, a “heterocyclic ring” may comprise one or more heteroatoms. In other embodiments, an aliphatic group may comprise or be substituted by one or more heteroatoms.

As used herein, horseradish peroxidase (HRP) is an enzyme that can be conjugated to a labeled molecule. It produces a colored, fluorimetric, or luminescent derivative of the labeled molecule when incubated with a proper substrate, allowing it to be detected and quantified. HRP acts in the presence of an electron donor to first form an enzyme substrate complex and then subsequently acts to oxidize an electronic donor. For example, HRP may act on 3,3′-diaminobenzidinetrahydrochloride (DAB) to produce a detectable color. HRP may also act upon a labeled tyramide conjugate, or tyramide like reactive conjugates (i.e., ferulate, coumaric, caffeic, cinnamate, dopamine, etc.), to deposit a colored or fluorescent or colorless reporter moiety for tyramide signal amplification (TSA).

As used herein, the term “label” refers to a detectable moiety that may be atoms or molecules, or a collection of atoms or molecules. A label may provide an optical, electrochemical, magnetic, or electrostatic (e.g., inductive, capacitive) signature which may be detected.

As used herein, the terms “multiplex,” “multiplexed,” or “multiplexing” refer to detecting multiple targets in a sample concurrently, substantially simultaneously, or sequentially. Multiplexing can include identifying and/or quantifying multiple distinct nucleic acids (e.g., DNA, RNA, mRNA, miRNA) and polypeptides (e.g., proteins) both individually and in any and all combinations.

As used herein, the term “nucleic acid molecule” or “polynucleotide” refers to a polymeric form of nucleotides of any length, either deoxyribonucleotides or ribonucleotides, or analogs thereof. Polynucleotides may have any three-dimensional structure, and may perform any function, known or unknown. Unless specifically limited, the terms encompass nucleic acids or polynucleotides including known analogues of natural nucleotides that have similar binding properties as the reference nucleic acid and are metabolized in a manner similar to naturally occurring nucleotides. Non-limiting examples of polynucleotides include coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the term “oligonucleotide,” refers to an oligomer of nucleotide or nucleoside monomer units wherein the oligomer optionally includes non-nucleotide monomer units, and/or other chemical groups attached at internal and/or external positions of the oligomer. The oligomer can be natural or synthetic and can include naturally-occurring oligonucleotides, or oligomers that include nucleosides with non-naturally-occurring (or modified) bases, sugar moieties, phosphodiester-analog linkages, and/or alternative monomer unit chiralities and isomeric structures (e.g., 5′- to 2′-linkage, L-nucleosides, α-anomer nucleosides, β-anomer nucleosides, locked nucleic acids (LNA), peptide nucleic acids (PNA)).

As used herein, the term “primary antibody” refers to an antibody which binds specifically to the target protein antigen in a tissue sample. A primary antibody is generally the first antibody used in an immunohistochemical procedure. In some embodiments, the primary antibody is a monoclonal antibody.

As used herein, the terms “reactive group” or “reactive functional group” refer to a functional group that are capable of chemically associating with, interacting with, hybridizing with, hydrogen bonding with, or coupling with a functional group of a different moiety. In some embodiments, a “reaction” between two reactive groups or two reactive functional groups may mean that a covalent linkage is formed between two reactive groups or two reactive functional groups; or may mean that the two reactive groups or two reactive functional groups associate with each other, interact with each other, hybridize to each other, hydrogen bond with each other, etc.

For example, the reactive group might be an amine-reactive group, such as an isothiocyanate, an isocyanate, an acyl azide, an NHS ester, an acid chloride, such as sulfonyl chloride, aldehydes and glycols, epoxides and oxiranes, carbonates, arylating agents, imidoesters, carbodiimides, anhydrides, and combinations thereof. Suitable thiol-reactive functional groups include haloacetyl and alkyl halides, maleimides, aziridines, acryloyl derivatives, arylating agents, thiol-disulfide exchange reagents, such as pyridyl disulfides, TNB-thiol, and disulfide reductants, and combinations thereof. Suitable carboxylate-reactive functional groups include diazoalkanes, diazoacetyl compounds, carbonyldiimidazole compounds, and carbondiimides. Suitable hydroxyl-reactive functional groups include epoxides and oxiranes, carbonyldiimidazole, N,N′-disuccinimidyl carbonates or N-hydroxysuccinimidyl chloroformates, periodate oxidizing compounds, enzymatic oxidation, alkyl halogens, and isocyanates. Aldehyde and ketone-reactive functional groups include hydrazines, Schiff bases, reductive amination products, Mannich condensation products, and combinations thereof. Active hydrogen-reactive compounds include diazonium derivatives, Mannich condensation products, iodination reaction products, and combinations thereof. Photoreactive chemical functional groups include aryl azides, halogenated aryl azides, benzophonones, diazo compounds, diazirine derivatives, and combinations thereof.

As used herein, the term “secondary antibody” herein refers to an antibody which binds specifically to a primary antibody, thereby forming a bridge between the primary antibody and a subsequent reagent (e.g., a label, an enzyme, etc.), if any. The secondary antibody is generally the second antibody used in an immunohistochemical procedure.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant that is at least 10⁻³ M greater, 10⁻⁴ M greater or 10⁻⁵ M greater than a binding constant for either of the two members of the binding pair with other molecules in a biological sample). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “substituted” is contemplated to include all permissible substituents of organic compounds. Whenever a group or moiety is described as being “substituted” or “optionally substituted” (or “optionally having” or “optionally comprising”) that group may be unsubstituted or substituted with one or more of the indicated substituents. Likewise, when a group is described as being “substituted or unsubstituted” if substituted, the substituent(s) may be selected from one or more the indicated substituents.

In some embodiments, the permissible substituents include acyclic and cyclic, branched and unbranched, carbocyclic and heterocyclic, and aromatic and nonaromatic substituents of organic compounds. In some embodiments, the permissible substituents can be one or more and the same or different for appropriate organic compounds. In some embodiments if no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, cyanate, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, ether, amino (e.g. a mono-substituted amino group or a di-substituted amino group), and protected derivatives thereof. Any of the above groups may include one or more heteroatoms, including O, N, or S. For example, where a moiety is substituted with an alkyl group, that alkyl group may comprise a heteroatom selected from O, N, or S (e.g. —(CH₂—CH₂—O—CH₂—CH₂)—).

As used herein, the term “target” refers to any molecule for which the presence, location and/or concentration is or can be determined. Examples of target molecules include proteins, nucleic acid sequences, and haptens, such as haptens covalently bonded to proteins. Target molecules are typically detected using one or more conjugates of a specific binding molecule and a detectable label.

As used herein, the terms “tyramide signal amplification” or “TSA” refer to an enzyme-mediated detection method that utilizes the catalytic activity of a peroxidase (such as horseradish peroxidase) to generate high-density labeling of a target molecule (such as a protein or nucleic acid sequence) in situ. TSA typically involves three basic steps: (1) binding of a specific binding member (e.g., an antibody, such as a monoclonal antibody) to the target followed by secondary detection of the specific binding member with a second peroxidase-labeled specific binding member; (2) activation of multiple copies of a labeled tyramide derivative (e.g., a hapten-labeled tyramide) by the peroxidase; and (3) covalent coupling of the resulting highly reactive tyramide radicals to residues (e.g., the phenol moiety of protein tyrosine residues) proximal to the peroxidase-target interaction site, resulting in deposition of haptens proximally (diffusion and reactivity mediated) to the target. In some examples of TSA, more or fewer steps are involved; for example, the TSA method can be repeated sequentially to increase signal. Methods of performing TSA and commercial kits and reagents for performing TSA are available (see, e.g., AmpMap Detection Kit with TSA™, Cat. No. 760-121, Ventana Medical Systems, Tucson, Ariz.; Invitrogen; TSA kit No. T-20911, Invitrogen Corp, Carlsbad, Calif.). Other enzyme-catalyzed, hapten or signaling linked reactive species can be alternatively used as they may become available.

As used herein, the symbol “ [symbol] ” refers to a location a moiety is bonded to another moiety.

Overview

The present disclosure is directed to “caged haptens,” conjugates comprising a specific binding entity and a “caged hapten,” and methods of using the same to detect one or more targets within a sample (e.g., one or more protein targets within the sample that are within close proximity to each other). As will be described in more detail herein, caged haptens or caged hapten-conjugates described herein facilitate the detection of protein dimers or proteins in close proximity to each other.

A “caged hapten” is a hapten whose structure has been modified such that a suitable anti-hapten antibody no longer recognizes the hapten and no binding event occurs. For instance, a DIG hapten that is coupled to a phosphate group may no longer be recognized by an anti-DIG antibody. In effect, the hapten's identity and/or function is “masked” or “protected.” To achieve this masking or protecting, the haptens of the present disclosure include an enzymatically cleavable caging group (also referred to as an enzymatically cleavable masking group).

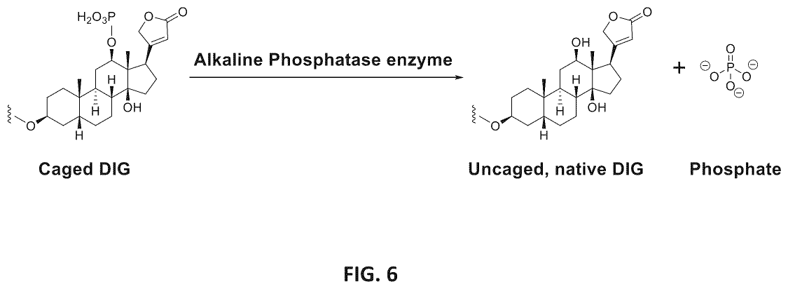

Upon introduction of an enzyme which acts on the enzymatically cleavable caging or masking group, the caging group or masking group is released to regenerate the “native” hapten (also referred to as an “uncaged hapten” or an “unmasked hapten.” This native hapten may then be recognized by the anti-hapten antibody. Thus, in the presence of an appropriate enzyme, the caged hapten is unmasked and an anti-hapten antibody is free to bind to it. In the example above, once the phosphate group of DIG is cleaved by an alkaline phosphatase enzyme, the native DIG hapten is revealed and an anti-DIG hapten is able to bind to it. FIG. 6 illustrates the unmasking of a caged hapten via enzymatic treatment to provide the native hapten, which is recognizable by an anti-hapten antibody.

Caged Haptens

In some embodiments, the caged haptens of the present disclosure have the structure of any one of Formulas (IA) and (IB):

R²—R¹—O-[DIG]-[Phosphoryl] (IA),

R²—R¹—O-[DIG]-PO₄H₂ (IB),

wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
[DIG] is digoxigenin or derived from [DIG];
[Phosphoryl] can be represented by the formula:

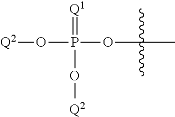

Q¹ is O or S;
Q² is H, —CH₃, or —CH₂CH₃;
where the group [Phosphoryl] or the group —PO₄H₂ may be attached to any position of [DIG].

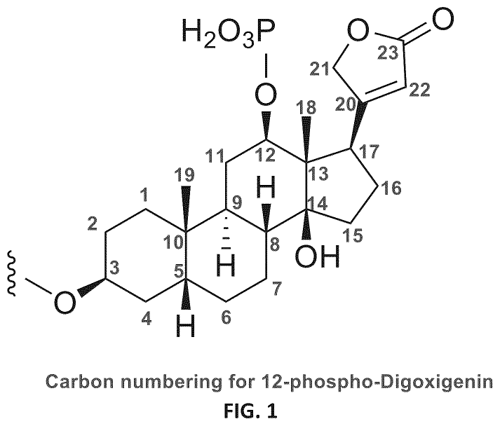

In some embodiments, [Phosphoryl] or —PO₄H₂ is coupled at the carbon 12 position of digoxigenin (see FIG. 1). In some embodiments, [Phosphoryl] or —PO₄H₂ is coupled at the carbon 12 position of a derivative or analog of digoxigenin.

In some embodiments, Q¹ is O. In some embodiments, Q¹ is O and at least one Q² is H. In some embodiments Q¹ is O and each Q² is H. In some embodiments, Q¹ is O and at least one Q² is —CH₃.

As noted above, in some embodiments, R¹ may a bond; or a group comprising a branched or unbranched, substituted or unsubstituted, saturated or unsaturated aliphatic group having between 1 and 30 carbon atoms, and optionally having one or more heteroatoms selected from the group consisting of O, N, or S. In other embodiments, R¹ may a bond; or a group comprising a branched or unbranched, substituted or unsubstituted, saturated or unsaturated aliphatic group having between 1 and 20 carbon atoms, and optionally having one or more heteroatoms selected from the group consisting of O, N, or S. In yet other embodiments, R¹ may a bond; or a group comprising a branched or unbranched, substituted or unsubstituted, saturated or unsaturated aliphatic group having between 5 and 15 carbon atoms, and optionally having one or more heteroatoms selected from the group consisting of O, N, or S. In further embodiments, R¹ may a bond; or a group comprising a branched or unbranched, substituted or unsubstituted, saturated or unsaturated aliphatic group having between 8 and 12 carbon atoms, and optionally having one or more heteroatoms selected from the group consisting of O, N, or S. In some embodiments, R¹ may comprise carbonyl, amine, ester, ether, amide, imine, thione or thiol groups. In other embodiments, R¹ may comprise one or more terminal groups selected from an amine, a carbonyl, ester, ether, amide, imine, thione, or thiol.

In some embodiments, R¹ is an unbranched aliphatic group having between 1 and 30 carbon atoms, and optionally including one or more heteroatoms selected from the group consisting of O, N, or S. In other embodiments, R¹ is an unbranched aliphatic group having between 1 and 20 carbon atoms, and optionally including one or more heteroatoms selected from the group consisting of O, N, or S. In yet other embodiments, R¹ is an unbranched aliphatic group having between 1 and 15 carbon atoms, and optionally including one or more heteroatoms selected from the group consisting of O, N, or S. In further embodiments, R¹ is an unbranched aliphatic group having between 1 and 12 carbon atoms, and optionally including one or more heteroatoms selected from the group consisting of O, N, or S. In even further embodiments, R¹ is an unbranched aliphatic group having between 1 and 8 carbon atoms, and optionally including one or more heteroatoms selected from the group consisting of O, N, or S. In yet even further embodiments, R¹ is an unbranched aliphatic group having between 4 and 8 carbon atoms, and optionally including one or more heteroatoms selected from the group consisting of O, N, or S.

In some embodiments, R¹ is an unbranched aliphatic group having between 1 and 30 carbon atoms, and optionally including one or more heteroatoms selected from the group consisting of O or N. In other embodiments, R¹ is an unbranched aliphatic group having between 1 and 20 carbon atoms, and optionally including one or more heteroatoms selected from the group consisting of O or N. In yet other embodiments, R¹ is an unbranched aliphatic group having between 1 and 15 carbon atoms, and optionally including one or more heteroatoms selected from the group consisting of O or N. In further embodiments, R¹ is an unbranched aliphatic group having between 1 and 12 carbon atoms, and optionally including one or more heteroatoms selected from the group consisting of O or N. In even further embodiments, R¹ is an unbranched aliphatic group having between 1 and 8 carbon atoms, and optionally including one or more heteroatoms selected from the group consisting of O or N. In yet even further embodiments, R¹ is an unbranched aliphatic group having between 4 and 8 carbon atoms, and optionally including one or more heteroatoms selected from the group consisting of O or N.

In some embodiments, R¹ is an unbranched aliphatic group having between 1 and 30 carbon atoms, and optionally including one or more oxygen heteroatoms. In other embodiments, R¹ is an unbranched aliphatic group having between 1 and 20 carbon atoms, and optionally including one or more oxygen heteroatoms. In yet other embodiments, R¹ is an unbranched aliphatic group having between 1 and 15 carbon atoms, and optionally including one or more oxygen heteroatoms. In further embodiments, R¹ is an unbranched aliphatic group having between 1 and 12 carbon atoms, and optionally including one or more oxygen heteroatoms. In even further embodiments, R¹ is an unbranched aliphatic group having between 1 and 8 carbon atoms, and optionally including one or more oxygen heteroatoms. In yet even further embodiments, R¹ is an unbranched aliphatic group having between 4 and 8 carbon atoms, and optionally including one or more oxygen heteroatoms.

In some embodiments, R¹ is an unbranched aliphatic group having between 1 and 30 carbon atoms, and optionally including one or more oxygen heteroatoms, and including at least one substitution on at least one carbon atom. In other embodiments, R¹ is an unbranched aliphatic group having between 1 and 20 carbon atoms, and optionally including one or more oxygen heteroatoms, and including at least one substitution on at least one carbon atom. In yet other embodiments, R¹ is an unbranched aliphatic group having between 1 and 15 carbon atoms, and optionally including one or more oxygen heteroatoms, and including at least one substitution on at least one carbon atom. In further embodiments, R¹ is an unbranched aliphatic group having between 1 and 12 carbon atoms, and optionally including one or more oxygen heteroatoms, and including at least one substitution on at least one carbon atom. In even further embodiments, R¹ is an unbranched aliphatic group having between 1 and 8 carbon atoms, and optionally including one or more oxygen heteroatoms, and including at least one substitution on at least one carbon atom. In yet even further embodiments, R¹ is an unbranched aliphatic group having between 4 and 8 carbon atoms, and optionally including one or more oxygen heteroatoms, and including at least one substitution on at least one carbon atom.

In some embodiments, R¹ has the structure depicted in Formula (IIIA):

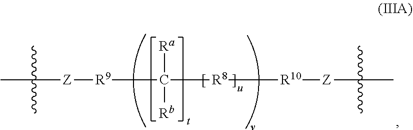

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

In some embodiments, v ranges from 1 to 6. In other embodiments, v ranges from 1 to 4. In yet other embodiments, v ranges from 2 to 6. In further embodiments, v ranges from 2-4.

In some embodiments, R⁸ is —C(R^{c})(R^{d})—, t+u is at least 2, and v is at least 1. In some embodiments, R⁸ is —C(R^{c})(R^{d})—, t+u is at least 2, and v is at least 2. In some embodiments, R⁸ is —C(R^{c})(R^{d})—, t+u is at least 2, and v is at least 3.

In some embodiments, R⁸ is —C(R^{c})(R^{d})—, at least one of R^{c} and R^{d} is H, t+u is at least 2, and v is at least 1. In some embodiments, R⁸ is —C(R^{c})(R^{d})—, at least one of R^{c} and R^{d} is H, t+u is at least 2, and v is at least 2. In some embodiments, R⁸ is —C(R^{c})(R^{d})—, at least one of R^{c} and R^{d} is H, t+u is at least 2, and v is at least 3.

In some embodiments, R⁸ is —C(R^{c})(R^{d})—, at least one of R^{c} and R^{d} is H, t+u is at least 2, v is at least 1, and at least one or R⁹ and R¹⁰ includes an amide group. In some embodiments, R⁸ is —C(R^{c})(R^{d})—, at least one of R^{c} and R^{d} is H, t+u is at least 2, v is at least 1, and at least one or R⁹ and R¹⁰ includes an amide group. In some embodiments, R⁸ is —C(R^{c})(R^{d})—, at least one of R^{c} and R^{d} is H, t+u is at least 2, v is at least 1, and at least one or R⁹ and R¹⁰ includes an amide group.

In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group. In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group. In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group.

In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different. In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different. In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different.

In some embodiments, R⁸ is O, t+u is at least 2, and v is at least 1. In some embodiments, R⁸ is O, t+u is at least 2, and v is at least 2. In some embodiments, R⁸ is O, t+u is at least 2, and v is at least 3.

In some embodiments, at least one of R^{a} and R^{b} is H, R⁸ is O, t+u is at least 2, v is at least 1, and at least one or R⁹ and R¹⁰ includes an amide group. In some embodiments, at least one of R^{a} and R^{b} is H, R⁸ is O, t+u is at least 2, v is at least 1, and at least one or R⁹ and R¹⁰ includes an amide group. In some embodiments, at least one of R^{a} and R^{b} is H, R⁸ is O, t+u is at least 2, v is at least 1, and at least one or R⁹ and R¹⁰ includes an amide group.

In some embodiments, at least one of R^{a} and R^{b} is H, R⁸ is O, t+u is at least 2, v is at least 1, at least one or R⁹ and R¹⁰ includes an amide group, and where both Z groups are different. In some embodiments, at least one of R^{a} and R^{b} is H, R⁸ is O, t+u is at least 2, v is at least 1, at least one or R⁹ and R¹⁰ includes an amide group, and where both Z groups are different. In some embodiments, at least one of R^{a} and R^{b} is H, R⁸ is O, t+u is at least 2, v is at least 1, at least one or R⁹ and R¹⁰ includes an amide group, and where both Z groups are different.

In other embodiments, R¹ has the structure depicted in Formula (IIIB):

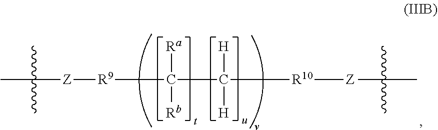

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, t+u is at least 2, v is at least 1, and at least one or R⁹ and R¹⁰ includes an amide group. In some embodiments, at least one of R^{a} and R^{b} is H, t+u is at least 2, v is at least 1, and at least one or R⁹ and R¹⁰ includes an amide group. In some embodiments, at least one of R^{a} and R^{b} is H, t+u is at least 2, v is at least 1, and at least one or R⁹ and R¹⁰ includes an amide group.

In some embodiments, at least one of R^{a} and R^{b} is H, t+u is at least 2, v is at least 1, at least one or R⁹ and R¹⁰ includes an amide group, and where both Z groups are different. In some embodiments, at least one of R^{a} and R^{b} is H, t+u is at least 2, v is at least 1, at least one or R⁹ and R¹⁰ includes an amide group, and where both Z groups are different. In some embodiments, at least one of R^{a} and R^{b} is H, t+u is at least 2, v is at least 1, at least one or R⁹ and R¹⁰ includes an amide group, and where both Z groups are different.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In some embodiments, at least one of R^{a} and R^{b} is H, u is 0, and at least one or R⁹ and R¹⁰ includes an amide group. In some embodiments, at least one of R^{a} and R^{b} is H, u is 0, v is at least 2, and at least one or R⁹ and R¹⁰ includes an amide group. In some embodiments, at least one of R^{a} and R^{b} is H, u is 0, v is at least 4, and at least one or R⁹ and R¹⁰ includes an amide group. In some embodiments, at least one of R^{a} and R^{b} is H, u is 0, v is at least 6, and at least one or R⁹ and R¹⁰ includes an amide group.

In other embodiments, R¹ has the structure depicted in Formula (IIIC):

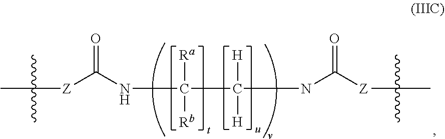

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In other embodiments, R¹ has the structure depicted in Formula (IIID):

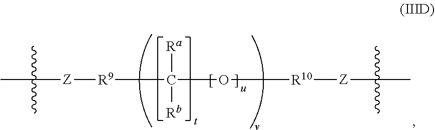

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In some embodiments, at least one of R^{a} and R^{b} is H, u is 0, and R⁹ is an amide. In some embodiments, at least one of R^{a} and R^{b} is H, u is 0, v is at least 2, and R⁹ is an amide. In some embodiments, at least one of R^{a} and R^{b} is H, u is 0, v is at least 4, and R⁹ is an amide. In some embodiments, at least one of R^{a} and R^{b} is H, u is 0, v is at least 6, and R⁹ is an amide.

In other embodiments, R¹ has the structure depicted in Formula (IIIE):

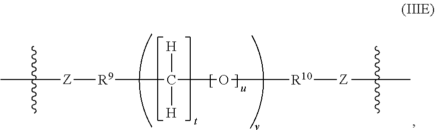

wherein
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—,
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4. In other embodiments, v is an integer ranging from 3 to 6. In yet other embodiments, v is an integer ranging from 4 to 6.

In some embodiments, R² is a carbonyl-reactive group. Suitable carbonyl-reactive groups include hydrazine, hydrazine derivatives, and amine.

In other embodiments, R² is an amine-reactive group. Suitable amine-reactive groups include active esters, such as NHS or sulfo-NHS, isothiocyanates, isocyanates, acyl azides, sulfonyl chlorides, aldehydes, glyoxals, epoxides, oxiranes, carbonates, aryl halides, imidoesters, anhydrides and the like.

In yet other embodiments, R² is a thiol-reactive group. Suitable thiol-reactive groups include non-polymerizable Michael acceptors, haloacetyl groups (such as iodoacetyl), alkyl halides, maleimides, aziridines, acryloyl groups, vinyl sulfones, benzoquinones, aromatic groups that can undergo nucleophilic substitution such as fluorobenzene groups (such as tetra and pentafluorobenzene groups), and disulfide groups such as pyridyl disulfide groups and thiols activated with Ellman's reagent.

In some embodiments, R² is a functional group or a moiety including a functional group capable of participating in a click chemistry reaction. “Click chemistry” is a chemical philosophy, independently defined by the groups of Sharpless and Meldal, that describes chemistry tailored to generate substances quickly and reliably by joining small units together. “Click chemistry” has been applied to a collection of reliable and self-directed organic reactions (Kolb, H. C.; Finn, M. G.; Sharpless, K. B. Angew). Chem. Int. Ed. 2001, 40, 2004-2021). For example, the identification of the copper catalyzed azide-alkyne [3+2] cycloaddition as a highly reliable molecular connection in water (Rostovtsev, V. V.; et al. Angew. Chem. Int. Ed. 2002, 41, 2596-2599) has been used to augment several types of investigations of biomolecular interactions (Wang, Q.; et al. J. Am. Chem. Soc. 2003, 125, 3192-3193; Speers, A. E.; et al. J. Am. Chem. Soc. 2003, 125, 4686-4687; Link, A. J.; Tirrell, D. A. J. Am. Chem. Soc. 2003, 125, 11164-11165; Deiters, A.; et al. J. Am. Chem. Soc. 2003, 125, 11782-11783). In addition, applications to organic synthesis (Lee, L. V.; et al. J. Am. Chem. Soc. 2003, 125, 9588-9589), drug discovery (Kolb, H. C.; Sharpless, K. B. Drug Disc. Today 2003, 8, 1128-1137; Lewis, W. G.; et al. Angew. Chem. Int. Ed. 2002, 41, 1053-1057), and the functionalization of surfaces (Meng, J.-C.; et al. Angew. Chem. Int. Ed. 2004, 43, 1255-1260; Fazio, F.; et al. J. Am. Chem. Soc. 2002, 124, 14397-14402; Collman, J. P.; et al. Langmuir 2004, ASAP, in press; Lummerstorfer, T.; Hoffmann, H. J. Phys. Chem. B 2004, in press) have also appeared.

In some embodiments, R² is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

In some embodiments, R¹ is a bond, or a group comprising a branched or unbranched, substituted or unsubstituted, saturated or unsaturated aliphatic group having between 1 and 20 carbon atoms, and optionally including one or more heteroatoms selected from the group consisting of O, N, or S. In other embodiments, R¹ is a bond, or a group comprising a branched or unbranched, substituted or unsubstituted, saturated or unsaturated aliphatic group having between 1 and 15 carbon atoms, and optionally including one or more heteroatoms selected from the group consisting of O, N, or S. In yet other embodiments, R¹ is a bond, or a group comprising a branched or unbranched, substituted or unsubstituted, saturated or unsaturated aliphatic group having between 1 and 12 carbon atoms, and optionally including one or more heteroatoms selected from the group consisting of O, N, or S. In yet other embodiments, R¹ is a bond, or a group comprising a branched or unbranched, substituted or unsubstituted, saturated or unsaturated aliphatic group having between 1 and 8 carbon atoms, and optionally including one or more heteroatoms selected from the group consisting of O, N, or S. In yet other embodiments, R¹ is a bond, or a group comprising a branched or unbranched, substituted or unsubstituted, saturated or unsaturated aliphatic group having between 4 and 8 carbon atoms, and optionally including one or more heteroatoms selected from the group consisting of O, N, or S.

In some embodiments, R¹ is an unbranched aliphatic group having between 1 and 30 carbon atoms, and optionally including one or more heteroatoms selected from the group consisting of O, N, or S. In other embodiments, R¹ is an unbranched aliphatic group having between 1 and 20 carbon atoms, and optionally including one or more heteroatoms selected from the group consisting of O, N, or S. In yet other embodiments, R¹ is an unbranched aliphatic group having between 1 and 15 carbon atoms, and optionally including one or more heteroatoms selected from the group consisting of O, N, or S. In further embodiments, R¹ is an unbranched aliphatic group having between 1 and 12 carbon atoms, and optionally including one or more heteroatoms selected from the group consisting of O, N, or S. In further embodiments, R¹ is an unbranched aliphatic group having between 1 and 8 carbon atoms, and optionally including one or more heteroatoms selected from the group consisting of O, N, or S. In further embodiments, R¹ is an unbranched aliphatic group having between 4 and 8 carbon atoms, and optionally including one or more heteroatoms selected from the group consisting of O, N, or S.

In some embodiments, R¹ is an unbranched aliphatic group having between 1 and 30 carbon atoms, and optionally including one or more heteroatoms selected from the group consisting of O or N. In other embodiments, R¹ is an unbranched aliphatic group having between 1 and 20 carbon atoms, and optionally including one or more heteroatoms selected from the group consisting of O or N. In yet other embodiments, R¹ is an unbranched aliphatic group having between 1 and 15 carbon atoms, and optionally including one or more heteroatoms selected from the group consisting of O or N. In further embodiments, R¹ is an unbranched aliphatic group having between 1 and 12 carbon atoms, and optionally including one or more heteroatoms selected from the group consisting of O or N. In further embodiments, R¹ is an unbranched aliphatic group having between 1 and 8 carbon atoms, and optionally including one or more heteroatoms selected from the group consisting of O or N. In further embodiments, R¹ is an unbranched aliphatic group having between 4 and 8 carbon atoms, and optionally including one or more heteroatoms selected from the group consisting of O or N.

In some embodiments, R¹ is an unbranched aliphatic group having between 1 and 30 carbon atoms, and optionally including one or more heteroatoms selected from the group consisting of O or N, and wherein R² is includes a thiol reactive group, a carbonyl reactive group, or an amine reactive group. In other embodiments, R¹ is an unbranched aliphatic group having between 1 and 20 carbon atoms, and optionally including one or more heteroatoms selected from the group consisting of O or N, and wherein R² is includes a thiol reactive group, a carbonyl reactive group, or an amine reactive group. In yet other embodiments, R¹ is an unbranched aliphatic group having between 1 and 15 carbon atoms, and optionally including one or more heteroatoms selected from the group consisting of O or N, and wherein R² is includes a thiol reactive group, a carbonyl reactive group, or an amine reactive group. In further embodiments, R¹ is an unbranched aliphatic group having between 1 and 12 carbon atoms, and optionally including one or more heteroatoms selected from the group consisting of O or N, and wherein R² is includes a thiol reactive group, a carbonyl reactive group, or an amine reactive group. In further embodiments, R¹ is an unbranched aliphatic group having between 1 and 8 carbon atoms, and optionally including one or more heteroatoms selected from the group consisting of O or N, and wherein R² is includes a thiol reactive group, a carbonyl reactive group, or an amine reactive group. In further embodiments, R¹ is an unbranched aliphatic group having between 4 and 8 carbon atoms, and optionally including one or more heteroatoms selected from the group consisting of O or N, and wherein R² is includes a thiol reactive group, a carbonyl reactive group, or an amine reactive group.

In some embodiments, R¹ is an unbranched aliphatic group having between 1 and 30 carbon atoms, and optionally including one or more heteroatoms selected from the group consisting of O or N, Q¹ is O, at least one Q² is H, and wherein R² is includes a thiol reactive group, a carbonyl reactive group, or an amine reactive group. In other embodiments, R¹ is an unbranched aliphatic group having between 1 and 20 carbon atoms, and optionally including one or more heteroatoms selected from the group consisting of O or N, Q¹ is O, at least one Q² is H, and wherein R² is includes a thiol reactive group, a carbonyl reactive group, or an amine reactive group. In yet other embodiments, R¹ is an unbranched aliphatic group having between 1 and 15 carbon atoms, and optionally including one or more heteroatoms selected from the group consisting of O or N, Q¹ is O, at least one Q² is H, and wherein R² is includes a thiol reactive group, a carbonyl reactive group, or an amine reactive group. In further embodiments, R¹ is an unbranched aliphatic group having between 1 and 12 carbon atoms, and optionally including one or more heteroatoms selected from the group consisting of O or N, Q¹ is O, at least one Q² is H, and wherein R² is includes a thiol reactive group, a carbonyl reactive group, or an amine reactive group. In further embodiments, R¹ is an unbranched aliphatic group having between 1 and 8 carbon atoms, and optionally including one or more heteroatoms selected from the group consisting of O or N, Q¹ is O, at least one Q² is H, and wherein R² is includes a thiol reactive group, a carbonyl reactive group, or an amine reactive group. In further embodiments, R¹ is an unbranched aliphatic group having between 4 and 8 carbon atoms, and optionally including one or more heteroatoms selected from the group consisting of O or N, Q¹ is O, at least one Q² is H, and wherein R² is includes a thiol reactive group, a carbonyl reactive group, or an amine reactive group.

In some embodiments, R¹ is an unbranched aliphatic group having between 1 and 30 carbon atoms, and optionally including one or more heteroatoms selected from the group consisting of O or N, and wherein R² is includes a group or moiety which includes a functional group capable of participating in a “click chemistry” reaction. In other embodiments, R¹ is an unbranched aliphatic group having between 1 and 20 carbon atoms, and optionally including one or more heteroatoms selected from the group consisting of O or N, and wherein R² is includes a group or moiety which includes a functional group capable of participating in a “click chemistry” reaction. In yet other embodiments, R¹ is an unbranched aliphatic group having between 1 and 15 carbon atoms, and optionally including one or more heteroatoms selected from the group consisting of O or N, and wherein R² is includes a group or moiety which includes a functional group capable of participating in a “click chemistry” reaction. In further embodiments, R¹ is an unbranched aliphatic group having between 1 and 12 carbon atoms, and optionally including one or more heteroatoms selected from the group consisting of O or N, and wherein R² is includes a group or moiety which includes a functional group capable of participating in a “click chemistry” reaction. Functional groups capable of participating in a “click chemistry” reaction are described herein. In further embodiments, R¹ is an unbranched aliphatic group having between 1 and 8 carbon atoms, and optionally including one or more heteroatoms selected from the group consisting of O or N, and wherein R² is includes a group or moiety which includes a functional group capable of participating in a “click chemistry” reaction. Functional groups capable of participating in a “click chemistry” reaction are described herein. In further embodiments, R¹ is an unbranched aliphatic group having between 4 and 8 carbon atoms, and optionally including one or more heteroatoms selected from the group consisting of O or N, and wherein R² is includes a group or moiety which includes a functional group capable of participating in a “click chemistry” reaction. Functional groups capable of participating in a “click chemistry” reaction are described herein.

In some embodiments, R¹ is an unbranched aliphatic group having between 1 and 30 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction. In other embodiments, R¹ is an unbranched aliphatic group having between 1 and 20 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction. In yet other embodiments, R¹ is an unbranched aliphatic group having between 1 and 15 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction. In further embodiments, R¹ is an unbranched aliphatic group having between 1 and 12 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction. In further embodiments, R¹ is an unbranched aliphatic group having between 1 and 8 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction. In further embodiments, R¹ is an unbranched aliphatic group having between 4 and 8 carbon atoms, and optionally including one or more heteroatoms selected from the group consisting of O or N, Q¹ is O, at least one Q² is H, and wherein R² is includes a group or moiety which includes a functional group capable of participating in a “click chemistry” reaction.

In some embodiments, R¹ is an unbranched aliphatic group having between 1 and 30 carbon atoms, and optionally including one or more oxygen heteroatoms. In other embodiments, R¹ is an unbranched aliphatic group having between 1 and 20 carbon atoms, and optionally including one or more oxygen heteroatoms. In yet other embodiments, R¹ is an unbranched aliphatic group having between 1 and 15 carbon atoms, and optionally including one or more oxygen heteroatoms. In further embodiments, R¹ is an unbranched aliphatic group having between 1 and 12 carbon atoms, and optionally including one or more oxygen heteroatoms. In further embodiments, R¹ is an unbranched aliphatic group having between 1 and 8 carbon atoms, and optionally including one or more oxygen heteroatoms. In further embodiments, R¹ is an unbranched aliphatic group having between 4 and 8 carbon atoms, and optionally including one or more oxygen heteroatoms.

In some embodiments, R¹ is an unbranched aliphatic group having between 1 and 30 carbon atoms, and optionally including one or more oxygen heteroatoms, and further including at least one substitution on one of the carbon atoms. In other embodiments, R¹ is an unbranched aliphatic group having between 1 and 20 carbon atoms, and optionally including one or more oxygen heteroatoms, and further including at least one substitution on one of the carbon atoms. In yet other embodiments, R¹ is an unbranched aliphatic group having between 1 and 15 carbon atoms, and optionally including one or more oxygen heteroatoms, and further including at least one substitution on one of the carbon atoms. In further embodiments, R¹ is an unbranched aliphatic group having between 1 and 12 carbon atoms, and optionally including one or more oxygen heteroatoms, and further including at least one substitution on one of the carbon atoms. In further embodiments, R¹ is an unbranched aliphatic group having between 1 and 8 carbon atoms, and optionally including one or more oxygen heteroatoms, and further including at least one substitution on one of the carbon atoms. In further embodiments, R¹ is an unbranched aliphatic group having between 4 and 8 carbon atoms, and optionally including one or more oxygen heteroatoms, and further including at least one substitution on one of the carbon atoms.

In some embodiments, R¹ is an unbranched aliphatic group having between 1 and 30 carbon atoms, and optionally including one or more oxygen heteroatoms, and wherein Q¹ is O, at least one Q² is H. In other embodiments, R¹ is an unbranched aliphatic group having between 1 and 20 carbon atoms, and optionally including one or more oxygen heteroatoms, and wherein Q¹ is O, at least one Q² is H. In yet other embodiments, R¹ is an unbranched aliphatic group having between 1 and 15 carbon atoms, and optionally including one or more oxygen heteroatoms, and wherein Q¹ is O, at least one Q² is H. In further embodiments, R¹ is an unbranched aliphatic group having between 1 and 12 carbon atoms, and optionally including one or more oxygen heteroatoms, and wherein Q¹ is O, at least one Q² is H. In further embodiments, R¹ is an unbranched aliphatic group having between 1 and 8 carbon atoms, and optionally including one or more oxygen heteroatoms, and wherein Q¹ is O, at least one Q² is H. In further embodiments, R¹ is an unbranched aliphatic group having between 4 and 8 carbon atoms, and optionally including one or more oxygen heteroatoms, and wherein Q¹ is O, at least one Q² is H.

In some embodiments, R¹ is a bond and R² is H. In some embodiments, R¹ is a bond, R² is H, and wherein Q¹ is O. In some embodiments, R¹ is a bond, R² is H, Q¹ is O, and at least one Q² is H.

In some embodiments, the caged haptens of the present disclosure have the structure of any one of Formulas (IIA)-(IIF):

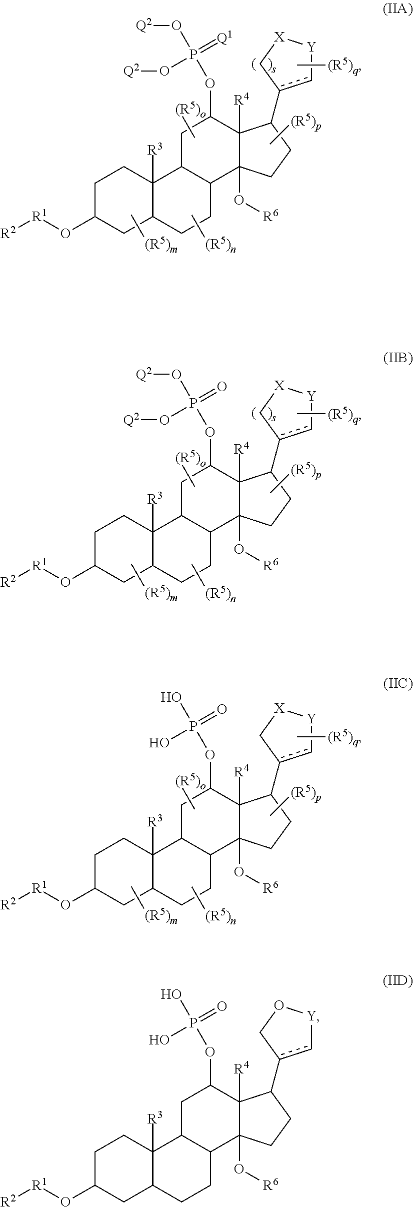

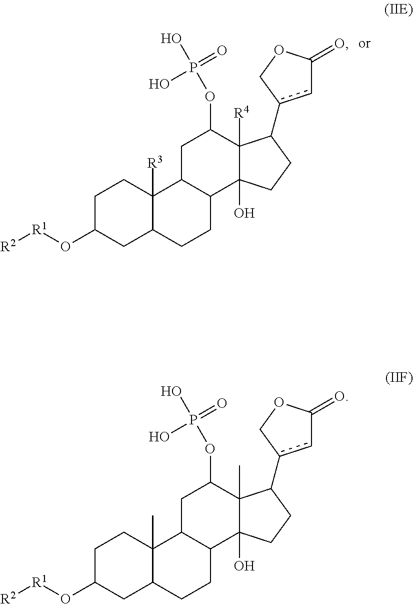

wherein
Q¹ is O or S;
Q² is H, —CH₃, or —CH₂CH₃;
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
each R⁵ is independently H, —CH₃, —CH₂CH₃, a halogen, or —C(O)H;
R⁶ is H or a linear or branched or substituted or unsubstituted C₁-C₆ alkyl group;
m, n, and o are each independently 0 or an integer ranging from 1 to 4;
p and q are each independently 0 or an integer ranging from 1 to 3;
s is 1 or 2; and
X and Y are each independently —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

As noted above, in some embodiments, R¹ may a bond; or a group comprising a branched or unbranched, substituted or unsubstituted, saturated or unsaturated aliphatic group having between 1 and 30 carbon atoms, and optionally having one or more heteroatoms selected from the group consisting of O, N, or S. In other embodiments, R¹ may a bond; or a group comprising a branched or unbranched, substituted or unsubstituted, saturated or unsaturated aliphatic group having between 1 and 20 carbon atoms, and optionally having one or more heteroatoms selected from the group consisting of O, N, or S. In yet other embodiments, R¹ may a bond; or a group comprising a branched or unbranched, substituted or unsubstituted, saturated or unsaturated aliphatic group having between 5 and 15 carbon atoms, and optionally having one or more heteroatoms selected from the group consisting of O, N, or S. In further embodiments, R¹ may a bond; or a group comprising a branched or unbranched, substituted or unsubstituted, saturated or unsaturated aliphatic group having between 6 and 12 carbon atoms, and optionally having one or more heteroatoms selected from the group consisting of O, N, or S. In further embodiments, R¹ may a bond; or a group comprising a branched or unbranched, substituted or unsubstituted, saturated or unsaturated aliphatic group having between 8 and 12 carbon atoms, and optionally having one or more heteroatoms selected from the group consisting of O, N, or S. In some embodiments, R¹ may comprise carbonyl, amine, ester, ether, amide, imine, thione or thiol groups. In other embodiments, R¹ may comprise one or more terminal groups selected from an amine, a carbonyl, ester, ether, amide, imine, thione, or thiol.

In some embodiments, R¹ is an unbranched aliphatic group having between 1 and 30 carbon atoms, and optionally including one or more heteroatoms selected from the group consisting of O, N, or S. In other embodiments, R¹ is an unbranched aliphatic group having between 1 and 20 carbon atoms, and optionally including one or more heteroatoms selected from the group consisting of O, N, or S. In yet other embodiments, R¹ is an unbranched aliphatic group having between 1 and 15 carbon atoms, and optionally including one or more heteroatoms selected from the group consisting of O, N, or S. In further embodiments, R¹ is an unbranched aliphatic group having between 1 and 12 carbon atoms, and optionally including one or more heteroatoms selected from the group consisting of O, N, or S. In further embodiments, R¹ is an unbranched aliphatic group having between 1 and 8 carbon atoms, and optionally including one or more heteroatoms selected from the group consisting of O, N, or S. In further embodiments, R¹ is an unbranched aliphatic group having between 4 and 8 carbon atoms, and optionally including one or more heteroatoms selected from the group consisting of O, N, or S.

In some embodiments, R¹ is an unbranched aliphatic group having between 1 and 30 carbon atoms, and optionally including one or more heteroatoms selected from the group consisting of O or N. In other embodiments, R¹ is an unbranched aliphatic group having between 1 and 20 carbon atoms, and optionally including one or more heteroatoms selected from the group consisting of O or N. In yet other embodiments, R¹ is an unbranched aliphatic group having between 1 and 15 carbon atoms, and optionally including one or more heteroatoms selected from the group consisting of O or N. In further embodiments, R¹ is an unbranched aliphatic group having between 1 and 12 carbon atoms, and optionally including one or more heteroatoms selected from the group consisting of O or N. In further embodiments, R¹ is an unbranched aliphatic group having between 1 and 8 carbon atoms, and optionally including one or more heteroatoms selected from the group consisting of O or N. In further embodiments, R¹ is an unbranched aliphatic group having between 4 and 8 carbon atoms, and optionally including one or more heteroatoms selected from the group consisting of O or N.

In some embodiments, R¹ is an unbranched aliphatic group having between 1 and 30 carbon atoms, and optionally including one or more oxygen heteroatoms. In other embodiments, R¹ is an unbranched aliphatic group having between 1 and 20 carbon atoms, and optionally including one or more oxygen heteroatoms. In yet other embodiments, R¹ is an unbranched aliphatic group having between 1 and 15 carbon atoms, and optionally including one or more oxygen heteroatoms. In further embodiments, R¹ is an unbranched aliphatic group having between 1 and 12 carbon atoms, and optionally including one or more oxygen heteroatoms. In further embodiments, R¹ is an unbranched aliphatic group having between 1 and 8 carbon atoms, and optionally including one or more oxygen heteroatoms. In further embodiments, R¹ is an unbranched aliphatic group having between 4 and 8 carbon atoms, and optionally including one or more oxygen heteroatoms.

In some embodiments, R¹ is an unbranched aliphatic group having between 1 and 30 carbon atoms, and optionally including one or more oxygen heteroatoms, and including at least one substitution on at least one carbon atom. In other embodiments, R¹ is an unbranched aliphatic group having between 1 and 20 carbon atoms, and optionally including one or more oxygen heteroatoms, and including at least one substitution on at least one carbon atom. In yet other embodiments, R¹ is an unbranched aliphatic group having between 1 and 15 carbon atoms, and optionally including one or more oxygen heteroatoms, and including at least one substitution on at least one carbon atom. In further embodiments, R¹ is an unbranched aliphatic group having between 1 and 12 carbon atoms, and optionally including one or more oxygen heteroatoms, and including at least one substitution on at least one carbon atom. In further embodiments, R¹ is an unbranched aliphatic group having between 1 and 8 carbon atoms, and optionally including one or more oxygen heteroatoms, and including at least one substitution on at least one carbon atom. In further embodiments, R¹ is an unbranched aliphatic group having between 4 and 8 carbon atoms, and optionally including one or more oxygen heteroatoms, and including at least one substitution on at least one carbon atom.

In some embodiments, R¹ has the structure depicted in Formula (IIIA):

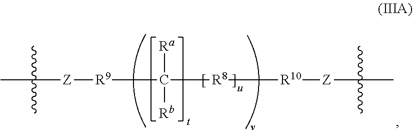

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, R⁸ is —C(R^{c})(R^{d})—, t+u is at least 2, and v is at least 1. In some embodiments, R⁸ is —C(R^{c})(R^{d})—, t+u is at least 2, and v is at least 2. In some embodiments, R⁸ is —C(R^{c})(R^{d})—, t+u is at least 2, and v is at least 3.

In some embodiments, R⁸ is —C(R^{c})(R^{d})—, at least one of R^{c} and R^{d} is H, t+u is at least 2, and v is at least 1. In some embodiments, R⁸ is —C(R^{c})(R^{d})—, at least one of R^{c} and R^{d} is H, t+u is at least 2, and v is at least 2. In some embodiments, R⁸ is —C(R^{c})(R^{d})—, at least one of R^{c} and R^{d} is H, t+u is at least 2, and v is at least 3.

In some embodiments, R⁸ is —C(R^{c})(R^{d})—, at least one of R^{c} and R^{d} is H, t+u is at least 2, v is at least 1, and at least one or R⁹ and R¹⁰ includes an amide group. In some embodiments, R⁸ is —C(R^{c})(R^{d})—, at least one of R^{c} and R^{d} is H, t+u is at least 2, v is at least 1, and at least one or R⁹ and R¹⁰ includes an amide group. In some embodiments, R⁸ is —C(R^{c})(R^{d})—, at least one of R^{c} and R^{d} is H, t+u is at least 2, v is at least 1, and at least one or R⁹ and R¹⁰ includes an amide group.

In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group. In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group. In some embodiments, at least one of R^{a} and R^{b} is H, R⁸ is —C(R^{c})(R^{d})—, at least one of R^{c} and R^{d} is H, t+u is at least 2, v is at least 1, and at least one or R⁹ and R¹⁰ includes an amide group.

In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different. In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different. In some embodiments, at least one of R^{a} and R^{b} is H, R⁸ is —C(R^{c})(R^{d})—, at least one of R^{c} and R^{d} is H, t+u is at least 2, v is at least 1, at least one or R⁹ and R¹⁰ includes an amide group, and where both Z groups are different.

In some embodiments, R⁸ is O, t+u is at least 2, and v is at least 1. In some embodiments, R⁸ is O, t+u is at least 2, and v is at least 2. In some embodiments, R⁸ is O, t+u is at least 2, and v is at least 3.

In some embodiments, at least one of R^{a} and R^{b} is H, R⁸ is O, t+u is at least 2, v is at least 1, and at least one or R⁹ and R¹⁰ includes an amide group. In some embodiments, at least one of R^{a} and R^{b} is H, R⁸ is O, t+u is at least 2, v is at least 1, and at least one or R⁹ and R¹⁰ includes an amide group. In some embodiments, at least one of R^{a} and R^{b} is H, R⁸ is O, t+u is at least 2, v is at least 1, and at least one or R⁹ and R¹⁰ includes an amide group.

In some embodiments, at least one of R^{a} and R^{b} is H, R⁸ is O, t+u is at least 2, v is at least 1, at least one or R⁹ and R¹⁰ includes an amide group, and where both Z groups are different. In some embodiments, at least one of R^{a} and R^{b} is H, R⁸ is O, t+u is at least 2, v is at least 1, at least one or R⁹ and R¹⁰ includes an amide group, and where both Z groups are different. In some embodiments, at least one of R^{a} and R^{b} is H, R⁸ is O, t+u is at least 2, v is at least 1, at least one or R⁹ and R¹⁰ includes an amide group, and where both Z groups are different.

In other embodiments, R¹ has the structure depicted in Formula (IIIB):

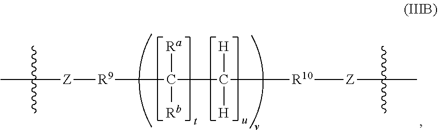

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, t+u is at least 2, v is at least 1, and at least one or R⁹ and R¹⁰ includes an amide group. In some embodiments, at least one of R^{a} and R^{b} is H, t+u is at least 2, v is at least 1, and at least one or R⁹ and R¹⁰ includes an amide group. In some embodiments, at least one of R^{a} and R^{b} is H, t+u is at least 2, v is at least 1, and at least one or R⁹ and R¹⁰ includes an amide group.

In some embodiments, at least one of R^{a} and R^{b} is H, t+u is at least 2, v is at least 1, at least one or R⁹ and R¹⁰ includes an amide group, and where both Z groups are different. In some embodiments, at least one of R^{a} and R^{b} is H, t+u is at least 2, v is at least 1, at least one or R⁹ and R¹⁰ includes an amide group, and where both Z groups are different. In some embodiments, at least one of R^{a} and R^{b} is H, t+u is at least 2, v is at least 1, at least one or R⁹ and R¹⁰ includes an amide group, and where both Z groups are different.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In some embodiments, at least one of R^{a} and R^{b} is H, u is 0, and at least one or R⁹ and R¹⁰ includes an amide group. In some embodiments, at least one of R^{a} and R^{b} is H, u is 0, v is at least 2, and at least one or R⁹ and R¹⁰ includes an amide group. In some embodiments, at least one of R^{a} and R^{b} is H, u is 0, v is at least 4, and at least one or R⁹ and R¹⁰ includes an amide group. In some embodiments, at least one of R^{a} and R^{b} is H, u is 0, v is at least 6, and at least one or R⁹ and R¹⁰ includes an amide group.

In other embodiments, R¹ has the structure depicted in Formula (IIIC):

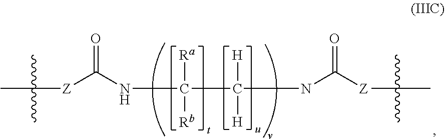

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In other embodiments, R¹ has the structure depicted in Formula (IIID):

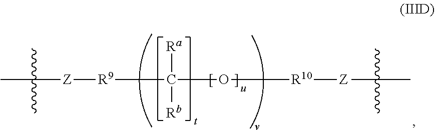

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8. In some embodiments, v ranges from 1 to 6. In other embodiments, v ranges from 1 to 4. In yet other embodiments, v ranges from 2 to 6. In further embodiments, v ranges from 2-4.

In some embodiments, at least one of R^{a} and R^{b} is H, and t+u is at least 2. In some embodiments, at least one of R^{a} and R^{b} is H, t+u is at least 2, and v is at least 2. In some embodiments, at least one of R^{a} and R^{b} is H, t+u is at least 2, and v is at least 3.

In some embodiments, at least one of R^{a} and R^{b} is H, and u is 0. In some embodiments, at least one of R^{a} and R^{b} is H, u is 0, and v is at least 2. In some embodiments, at least one of R^{a} and R^{b} is H, u is 0, and v is at least 4. In some embodiments, at least one of R^{a} and R^{b} is H, u is 0, and v is at least 6.

In some embodiments, at least one of R^{a} and R^{b} is H, u is 0, and R⁹ is an amide. In some embodiments, at least one of R^{a} and R^{b} is H, u is 0, v is at least 2, and R⁹ is an amide. In some embodiments, at least one of R^{a} and R^{b} is H, u is 0, v is at least 4, and R⁹ is an amide. In some embodiments, at least one of R^{a} and R^{b} is H, u is 0, v is at least 6, and R⁹ is an amide.

In other embodiments, R¹ has the structure depicted in Formula (IIIE):

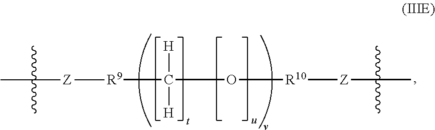

wherein
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—,
u and t are each independently 0, 1, or 2, provided that u+t is at least 1; and
v is an integer ranging from 1 to 8.

In some embodiments, v ranges from 1 to 6. In other embodiments, v ranges from 1 to 4. In yet other embodiments, v ranges from 2 to 6. In further embodiments, v ranges from 2-4. In other embodiments, v is an integer ranging from 3 to 6. In yet other embodiments, v is an integer ranging from 4 to 6.

In some embodiments, R² is a carbonyl-reactive group. Suitable carbonyl-reactive groups include hydrazine, hydrazine derivatives, and amine. In other embodiments, R² is an amine-reactive group. Suitable amine-reactive groups include active esters, such as NHS or sulfo-NHS, isothiocyanates, isocyanates, acyl azides, sulfonyl chlorides, aldehydes, glyoxals, epoxides, oxiranes, carbonates, aryl halides, imidoesters, anhydrides and the like. In yet embodiments, R² is a thiol-reactive group. Suitable thiol-reactive groups include non-polymerizable Michael acceptors, haloacetyl groups (such as iodoacetyl), alkyl halides, maleimides, aziridines, acryloyl groups, vinyl sulfones, benzoquinones, aromatic groups that can undergo nucleophilic substitution such as fluorobenzene groups (such as tetra and pentafluorobenzene groups), and disulfide groups such as pyridyl disulfide groups and thiols activated with Ellman's reagent.

In some embodiments, R² is a functional group or a moiety including a functional group capable of participating in a click chemistry reaction. In some embodiments, R² is a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

In some embodiments, at least one of R³ and R⁴ is —CH₃. In some embodiments, at least one of R³ and R⁴ is —CH₃; and R⁶ is a C₁-C₄ alkyl group. In some embodiments, at least one of R³ and R⁴ is —CH₃; and R⁶ is a C₁-C₂ alkyl group. In some embodiments, at least one of R³ and R⁴ is —CH₃; and R⁶ is H.

In some embodiments, both of R³ and R⁴ are —CH₃; and R⁶ is a C₁-C₄ alkyl group. In some embodiments, both of R³ and R⁴ are —CH₃; and R⁶ is a C₁-C₂ alkyl group. In some embodiments, at least both of R³ and R⁴ are —CH₃; and R⁶ is H.

In some embodiments, m, n, p, and q are each 1; and each R⁵ is selected from H or —CH₃. In some embodiments, m, n, p, and q are each 1; each R⁵ is selected from H or —CH₃; and at least one R³ or R⁴ is —CH₃. In some embodiments, m, n, p, and q are each 1; each R⁵ is selected from H or —CH₃; and both R³ and R⁴ are —CH₃.

In some embodiments, m, n, p, and q are each 1; each R⁵ is selected from H or —CH₃, and s is 1. In some embodiments, m, n, p, and q are each 1; each R⁵ is selected from H or —CH₃, s is 1; and at least one R³ or R⁴ is —CH₃. In some embodiments, m, n, p, and q are each 1; each R⁵ is selected from H or —CH₃, s is 1; and both R³ and R⁴ are —CH₃.

In some embodiments, m, n, p, and q are each 1; and at least one R⁵ is —CH₃. In some embodiments, m, n, p, and q are each 0. In some embodiments, m, n, p, and q are each 0; and at least one R³ or R⁴ is —CH₃. In some embodiments, m, n, p, and q are each 0; and both R³ are R⁴ is —CH₃.

In some embodiments, X is O. In some embodiments, X is O and Y is —C(O)—. In some embodiments, X is O. In some embodiments, X is O, Y is —C(O)—, and s is 1. In some embodiments, X is O, Y is —C(O)—, s is 1, and Q¹ is O. In some embodiments, X is O, Y is —C(O)—, s is 1, and Q¹ is S. In some embodiments, X is O, Y is —C(O)—, s is 1, Q¹ is O, and each Q² is H.

Non-limiting examples of the compounds of Formulas (IIIA) to (IIIF) include, but are not limited to:

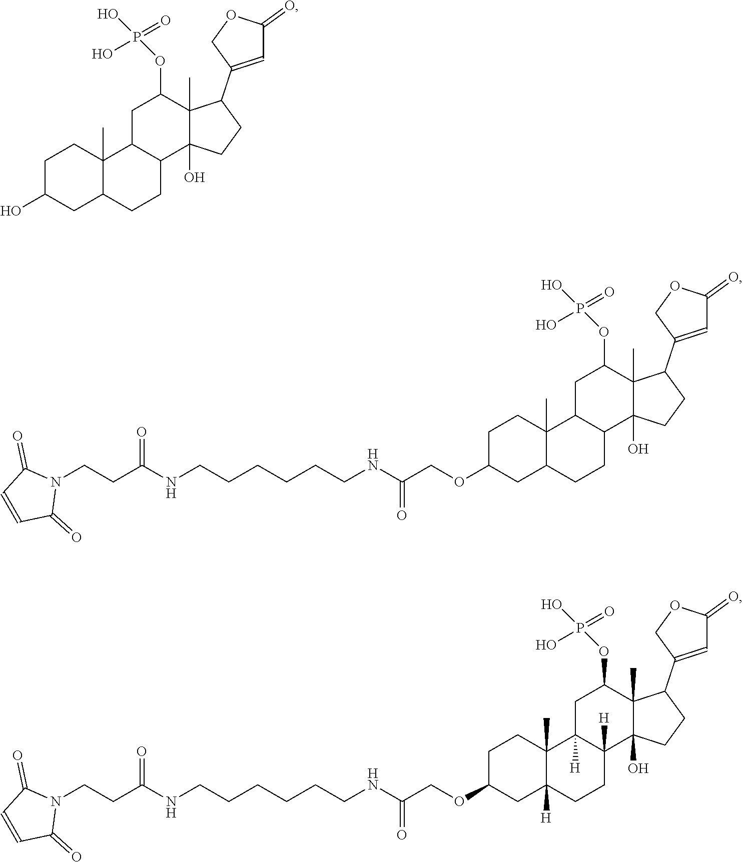

Caged Hapten Conjugates

The present disclosure also provides conjugates including a caged hapten. In some embodiments, the conjugates include a specific binding entity and a caged hapten, such as a caged hapten having the structure of any one of Formulas (IA), (IB), or (IIA)-(IIF). Methods of coupling a specific binding entity, e.g., an antibody, a nucleic acid molecule, an oligonucleotide, etc., to a caged hapten are described herein.

In some embodiments, the conjugates comprise an antibody (e.g., a primary antibody or a secondary antibody) coupled to a caged hapten having the structure of Formula (IA). In other embodiments, the conjugates comprise an antibody (e.g., a primary antibody or a secondary antibody) coupled to a caged hapten having the structure of Formula (IB). In other embodiments, the conjugates comprise an antibody (e.g., a primary antibody or a secondary antibody) coupled to a caged hapten having the structure of Formula (IIA). In other embodiments, the conjugates comprise an antibody (e.g., a primary antibody or a secondary antibody) coupled to a caged hapten having the structure of Formula (IIB). In other embodiments, the conjugates comprise an antibody (e.g., a primary antibody or a secondary antibody) coupled to a caged hapten having the structure of Formula (IIC). In other embodiments, the conjugates comprise an antibody (e.g., a primary antibody or a secondary antibody) coupled to a caged hapten having the structure of Formula (IID). In other embodiments, the conjugates comprise an antibody (e.g., a primary antibody or a secondary antibody) coupled to a caged hapten having the structure of Formula (IIE). In other embodiments, the conjugates comprise an antibody (e.g., a primary antibody or a secondary antibody) coupled to a caged hapten having the structure of Formula (IIF). In some embodiment, the antibody is a monoclonal antibody. In some embodiments, the primary or secondary antibody is a monoclonal antibody.

Examples of primary antibodies include anti-Her2, anti-Her3, anti-PD-L1, anti-PD-1, anti-E-Cadherin, anti-Beta-Catenin, anti-EGFR(Her1), anti-cMET, anti-GRB2, anti-TIGIT, anti-phosphotyrosine, anti-ubiquitin. Examples of secondary antibodies include anti-rabbit, anti-mouse, anti-rat, anti-goat, anti-camelid, anti-DIG, anti-DNP, anti-fluorescein.

The caged haptens of the present disclosure may be coupled to any portion of an antibody or any portion of a monoclonal antibody. The skilled artisan will appreciate that antibodies include three different types of functional groups suitable for covalent modifications, including (i) amines (—NH2), (ii) thiol groups (—SH), and (iii) carbohydrate residues. As such, any of the caged haptens disclosed herein may be coupled to amine residues, thiol residues, and carbohydrate residues or any combination thereof. In some embodiments, the caged haptens are coupled to Fc portions of the antibody.

In some embodiments, the specific binding entity is a nucleic acid molecule or an oligonucleotide. In some embodiments, the nucleic acid molecule comprises between 5 and about 50 nucleotides. In other embodiments, the nucleic acid molecule comprises between 5 and about 40 nucleotides. In other embodiments, the nucleic acid molecule comprises between 5 and about 30 nucleotides. In other embodiments, the nucleic acid molecule comprises between 5 and about 25 nucleotides. In other embodiments, the nucleic acid molecule comprises between 5 and about 20 nucleotides. In other embodiments, the nucleic acid molecule comprises between 5 and about 15 nucleotides.

In some embodiments, the caged hapten conjugates of the present disclosure have the structure of any one of Formulas (IVA) or (IVB):

[Specific Binding Entity]-W¹—W²—R¹—O-[DIG]-[Phosphoryl] (IVA),

[Specific Binding Entity]-W¹—W²—R¹—O-[DIG]-PO₄H₂ (IVB),

wherein
[Specific Binding Entity] is a specific binding entity;
W¹ is a bond, or a group comprising a branched or unbranched, substituted or unsubstituted, saturated or unsaturated aliphatic group having between 1 and 10 carbon atoms, and optionally including one or more heteroatoms selected from the group consisting of O, N, or S;
   W² is a bond or is derived from a reactive functional group; and
“reactive functional group,” R¹, [DIG], and [Phosphoryl] are as described herein.

In some embodiments, [Specific Binding Entity] is an antibody, e.g., a monoclonal antibody. In some embodiments, [Specific Binding Entity] is a primary antibody (e.g., a caged hapten conjugated to an antibody specific for Beta-Catenin). In some embodiments, [Specific Binding Entity] is a secondary antibody (e.g., a caged hapten conjugated to an antibody specific for an anti-Beta-Catenin antibody). In some embodiments, [Specific Binding Entity] is a nucleic acid molecule or an oligonucleotide.

In some embodiments, the caged hapten conjugates have the structure of any one of Formulas (VA) or (VF):

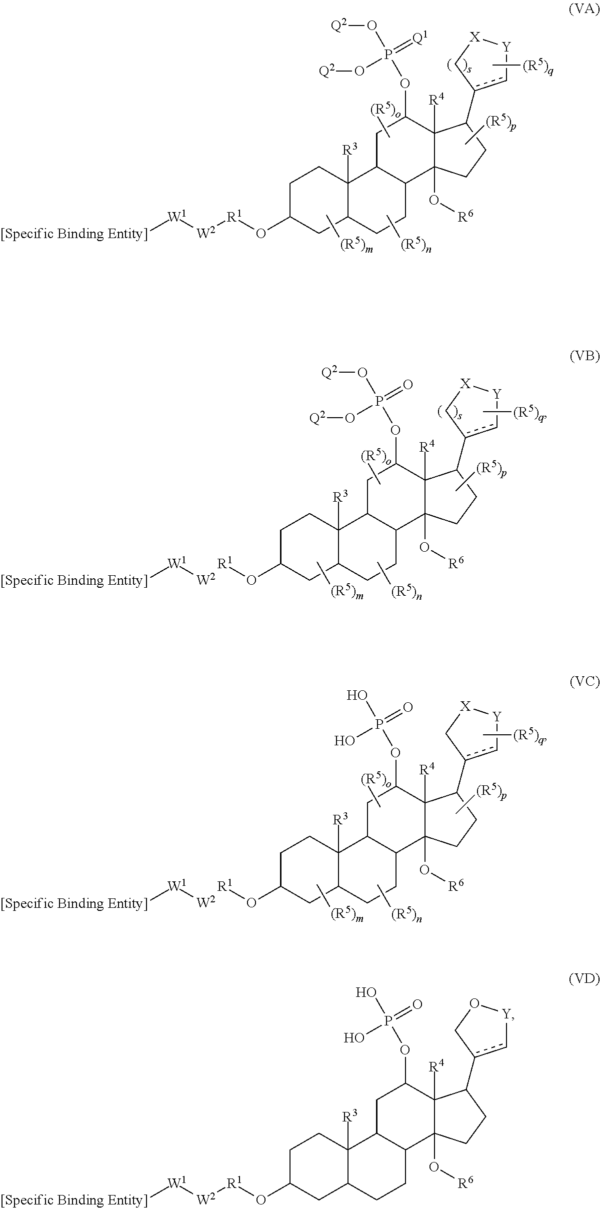

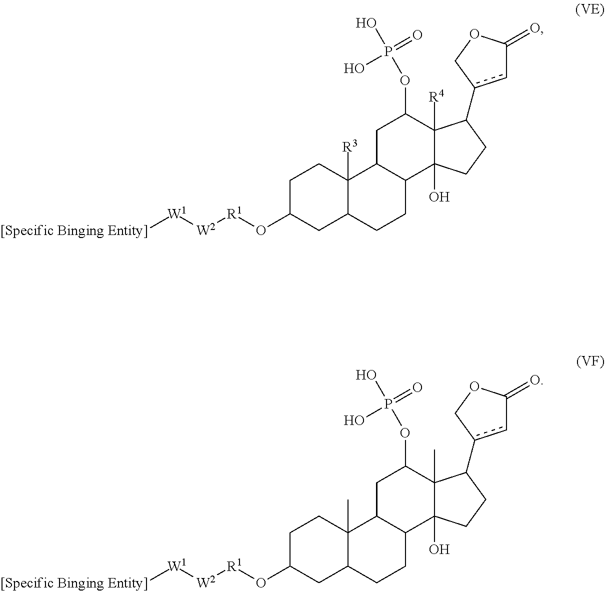

wherein

[Specific Binding Entity] is a specific binding entity;
W¹ is a bond, or a group comprising a branched or unbranched, substituted or unsubstituted, saturated or unsaturated aliphatic group having between 1 and 10 carbon atoms, and optionally including one or more heteroatoms selected from the group consisting of O, N, or S;
W² is a bond or derived from a reactive functional group (as described herein);
Q¹, Q², R¹, R³-R⁷, m, n, o, p, q, s, X, and Y are as defined herein.

In some embodiments, [Specific Binding Entity] is an antibody, e.g., a monoclonal antibody. In some embodiments, [Specific Binding Entity] is a primary antibody (e.g., a caged hapten conjugated to an antibody specific for Beta-Catenin). In some embodiments, [Specific Binding Entity] is a secondary antibody (e.g., a caged hapten conjugated to an antibody specific for an anti-Beta-Catenin antibody). In some embodiments, [Specific Binding Entity] is a nucleic acid molecule or an oligonucleotide.

Non-limiting examples of W¹ and W² groups are set forth below:

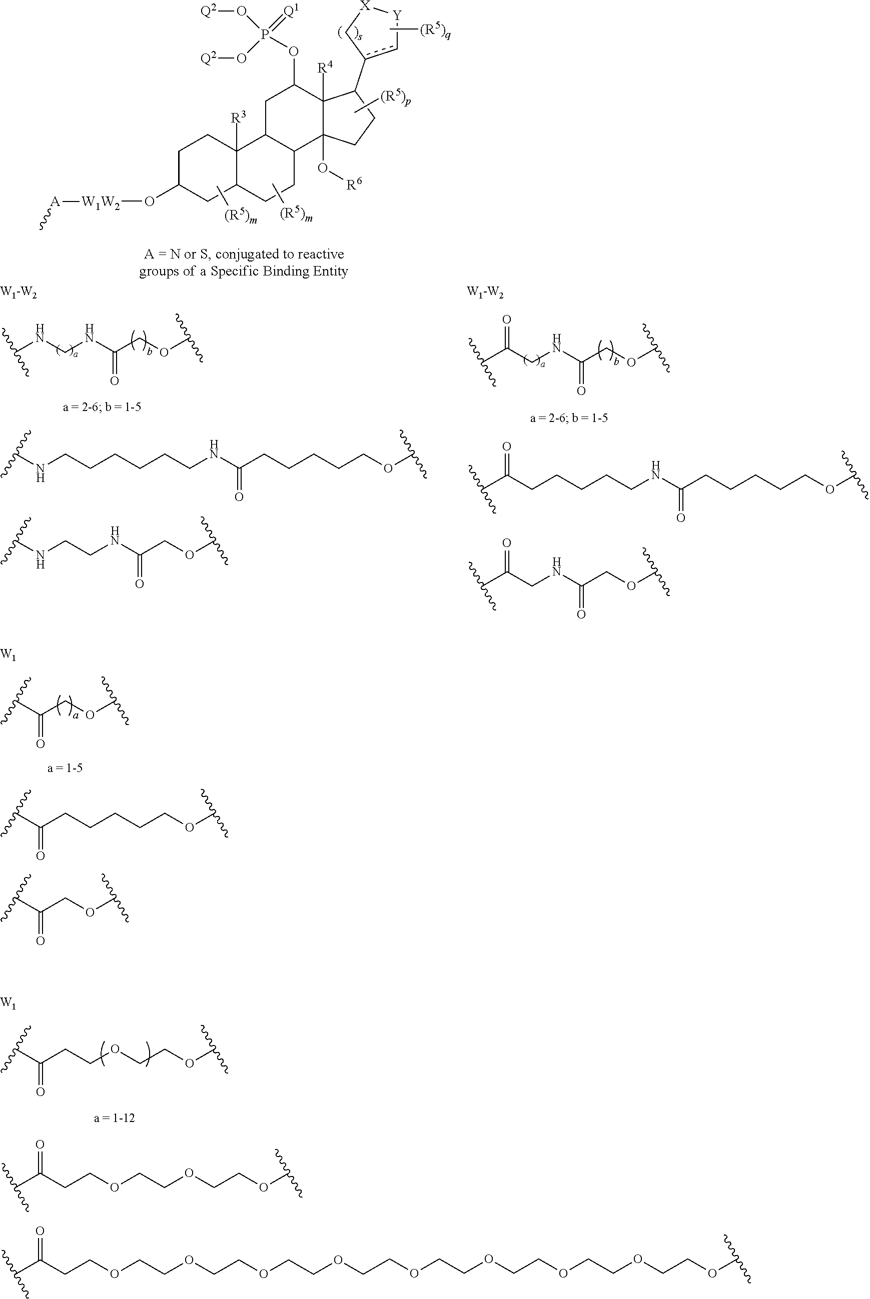

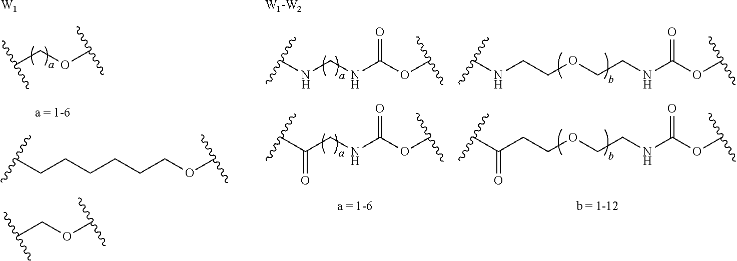

Non-limiting examples of conjugates of the present disclosure include:

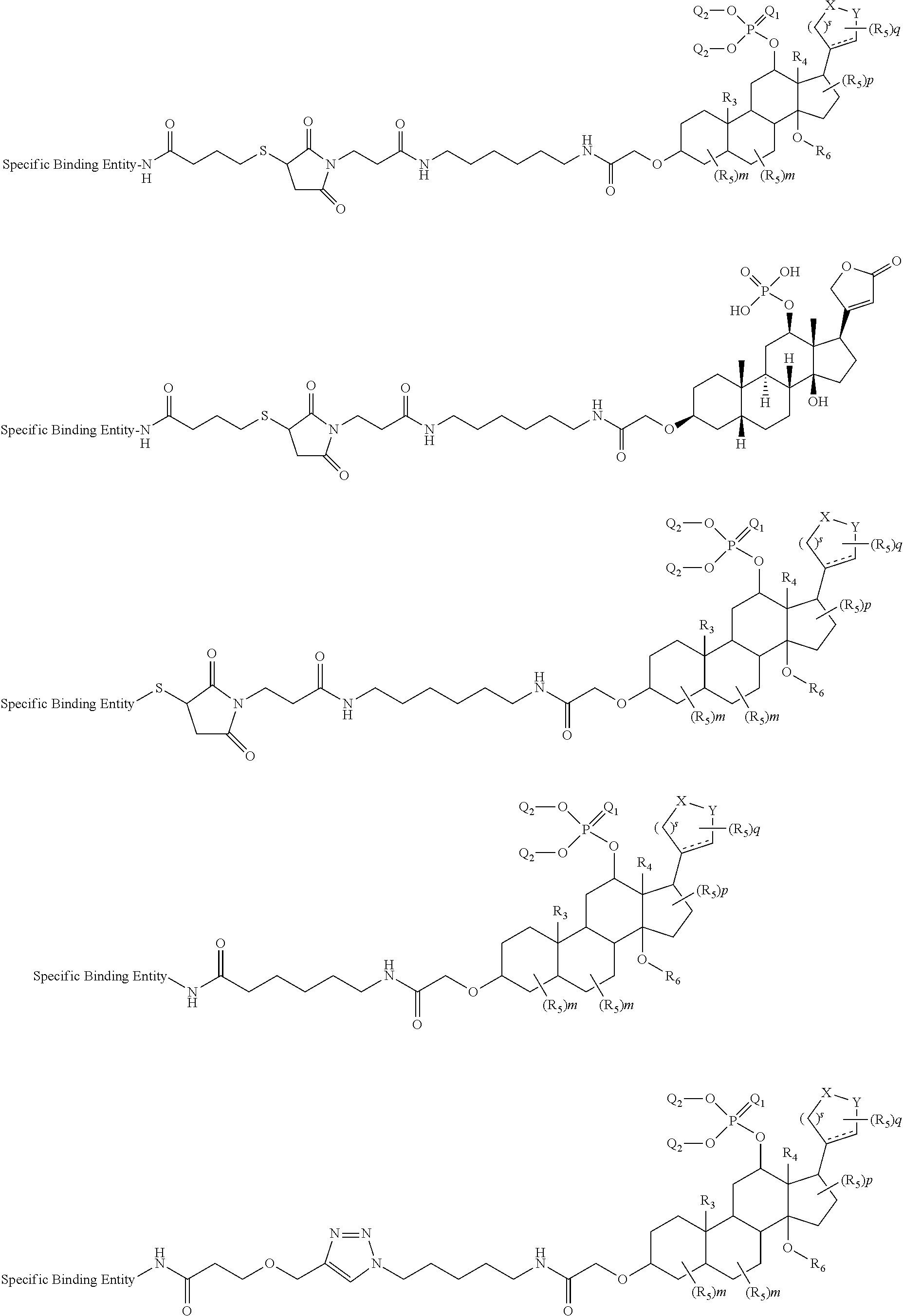

Synthesis of Caged Hapten Conjugates

The caged hapten conjugates of the present disclosure may be synthesized according to any method known to those of ordinary skill in the art. In some embodiments, a caged hapten (such as any of those described herein, including any of those of Formulas (IA), (IB), and (IIA)-(IIF)) may be conjugated to a thiol group of an antibody, e.g., a thiol group of a monoclonal antibody. In some embodiments, thiol groups are first introduced to the antibody by treating the antibody with a reducing agent such as dithiothreitol (DTT) or dithioerythritol (DTE). For a mild reducing agent, such as DTE or DTT, a concentration of between about 1 mM and about 40 mM (for example, a concentration of between about 5 mM and about 30 mM or between about 15 mM and about 25 mM) is utilized to introduce a limited number of thiols (such as between about 2 and about 6) to the antibody, while keeping the antibody intact (which can be determined by size-exclusion chromatography). Following treatment with the reducing agent, an excess of a caged hapten bearing a thiol reactive group (e.g., a maleimide group) is introduced to form the respective caged hapten-antibody conjugate. Other methods of introducing one or more thiol groups are described in United States Patent Publication No. 2016/0187324, the disclosure of which is hereby incorporated by reference herein in its entirety.

In other embodiments, a caged hapten may be conjugated to a Fc portion of an antibody. In some embodiments, an Fc portion of an antibody is first oxidized to form an aldehyde and the caged hapten is subsequently coupled to the oxidized Fc portion of the antibody through a reactive functional group on the caged hapten (e.g., with a carbonyl-reactive group, such as hydrazide group).

In yet other embodiments, a caged hapten may be conjugated to a lysine residue of an antibody, e.g., a lysine residue of a monoclonal antibody. As illustrated in the synthetic scheme which follows (Scheme 2), in some embodiments, the antibody is first treated with an excess of Traut's reagent (2-iminothiolane hydrochloride) before adding an excess of an appropriately functionalized caged hapten (e.g., one bearing a thiol reactive group, such as a maleimide group).

Following synthesis of the caged hapten conjugates, the conjugates may be purified, such as by size exclusion chromatography (SEC), and then characterized, such as by gel electrophoresis and/or UV-Vis.

Proximity Detection Using Caged Hapten Conjugates

As will be described in more detail herein, the present disclosure facilitates the detection of protein-protein complexes, e.g., protein dimers or proteins in close proximity to each other (e.g., those having a proximity of 5000 nm or less). In some embodiments, the assay is able to detect protein dimers or proteins having a proximity of 4000 nm or less. In other embodiments, the assay is able to detect protein dimers or proteins having a proximity of 3000 nm or less. In other embodiments, the assay is able to detect protein dimers or proteins having a proximity of 2500 nm or less. In other embodiments, the assay is able to detect protein dimers or proteins having a proximity of 2000 nm or less. In other embodiments, the assay is able to detect protein dimers or proteins having a proximity of 1500 nm or less. In yet other embodiments, the assay is able to detect protein dimers or proteins having a proximity of 1000 nm or less. In further embodiments, the assay is able to detect protein dimers or proteins having a proximity of 500 nm or less.

Protein-protein complexes (PPCs) form signaling nodes and hubs of molecular networks in all physiological processes, including cellular disease states and cancer. The reprogrammed cancer-initiating cells acquire and maintain all characteristics of cancer by gaining new physical and molecular features and altering molecular signaling pathways leading to pathological outcomes. It is believed that PPCs are responsible for transmitting oncogenic signals in those cells. It is also believed that PPCs participate in proliferating signaling and evasion of growth suppressors, and as a result lead to the development and progression of cancer. Non-limiting examples of protein-protein complexes include any of the Her1/2/3/4 proteins with each other; PD-1 with PD-L1; and/or PD-L2, EGFR (Her1) with any of it associated ligands (AREG, EREG); adaptor protein GRB2 with phosphorylated tyrosine proteins such as EGFR, cMET, Her2, MUC1; TIGIT with CD155.

Applicant submits that PPCs represent a highly promising class of targets for therapeutic development, as well as for functional diagnostics in immunohistochemistry (IHC). Traditional IHC detects the presence of single epitopes with a resolution limit in the range of 200 nanometers due to the diffraction limit of conventional light microscopy. As a result, it is only possible to describe the proteins in terms of co-localization, rather than complexes that occur on the order of tens of nanometers. The ability to interrogate for the presence and distribution of specific intermolecular complexes on frozen and paraffin embedded tissue allows for IHC to transition from structural to functional diagnostics. The definition of a biomarker therefore becomes broader in including PPCs and the molecular networks within the human interactome that they represent.

It is believed that the disclosed proximity assay is more general than merely measuring protein-protein interactions. Indeed, the disclosed assay allows for the measurement of the proximity of binding moieties. In practice, the binding moieties (e.g., antibodies) may be directed against targets with minimal or no distance between them. Examples of this could include signaling events like phosphorylation of proteins. In this case, if one antibody is directed against an epitope on a protein (e.g., HER2), and a second antibody is directed against all phospho-tyrosines, then the proximity signal would represent all the phosphorylated HER2 proteins. This type of assay is more binary (yes/no) than pairs of proteins that interact with each other.

Any of the caged hapten-conjugates of the present disclosed may be used in both (i) simplex assays for the detection of protein dimers or protein proximity; and (ii) multiplex assays for the detection of protein dimers or protein proximity and detection of total protein. “Total protein” refers to the normal IHC visualization of any given protein, whereas a proximity signal is the portion of this protein that is involved in a given interaction. For example, and in the case of a PD-1/PD-L1 assay, the proximity signal would visualize only the interaction between PD-1 and PD-L1, whereas the total protein signal would visualize all PD-1 in the sample. Expressing the score for proximity as a numerator and the score for total protein as a denominator could give the fraction or percentage of PD-1 that is involved in an interaction. This may be important as a diagnostic for detecting active pharmaceutical ingredients that disturb protein-protein interaction where the expression of protein is less important that the number of interacting proteins. This is believed to hold true for phosphorylation, as described above, where instead of just receiving an arbitrary score for the phosphorylated signal, one may be able to quantify what percentage of a give protein is phosphorylated.

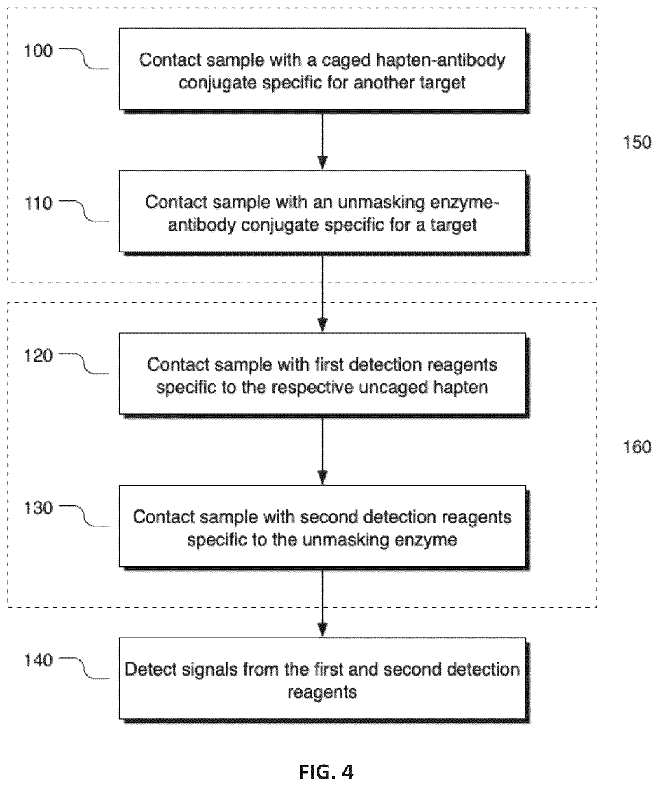

With reference to FIG. 4, the detection of protein dimers takes place in two general stages. In a first stage 150, a sample is labeled with at least two different types of antibody conjugates, e.g., at least two different types of monoclonal antibody conjugates. In a second stage 160, the sample is contacted with a first set of detection reagents (e.g., for simplex assays), and optionally a second set of detection reagents (e.g., for multiplex assays). Following the second stage 160, signals from the first and optionally the second sets of detection reagents are detected (step 140). The signals may be detected according to methods known to those of ordinary skill in the art, such as those described in U.S. Pat. No. 10,041,950, and in U.S. Publication Nos. 2019/0204330, 2017/0089911, and 2019/0187130 and in PCT Publication No. WO/2014/143155, the disclosures of which are hereby incorporated by reference herein in its entirety.

In the first stage 150, a sample is contacted with a caged hapten-antibody conjugate specific for a first target to form a first target-caged hapten-antibody conjugate complex (step 100). In some embodiments, the caged-hapten-antibody conjugate has any one of Formulas (IVA), (IVB), or (VA)-(VF). As described further herein, the caged hapten portion of the caged hapten-antibody conjugate is capable of becoming unmasked to provide the respective unmasked hapten, i.e., the “native hapten” or the “uncaged hapten.” For example, a caged DIG hapten may be unmasked to provide the native DIG hapten. Likewise, a caged steroid may be unmasked to provide the native steroid.

Subsequently, the sample is first contacted with an unmasking enzyme-antibody conjugate specific for a second target to form a second target-unmasking enzyme-antibody conjugate complex (step 110). In some embodiments, the unmasking enzyme (e.g., a phosphatase, a phosphodiesterase, a phosphotriesterase) of the unmasking enzyme-antibody conjugate is reactive with an enzyme substrate portion of the caged hapten-antibody conjugate introduced at step 100. For example, the unmasking enzyme of the unmasking enzyme-antibody conjugate may be reactive with either the [Phosphoryl] group of Formula (IVA), the PO₄H₂ group of Formula (IVB), or the phosphate-containing group of any one of Formulas (VA)-(VF).

In some embodiments, steps 100 and 110 may be performed in any order or may be performed simultaneously. In some embodiments, step 100 is performed first, and step 110 is performed second. In other embodiments, step 110 is performed first, and step 100 is performed second.

In some embodiments, the first stage 150 also includes one or more “decaging steps” where on-slide conditions are changed to enhance enzyme activity. “Decaging steps” include, but are not limited to, one or more washing steps or steps to adjust the pH (e.g., a pH ranging from about 7 to about 8.5). In some embodiments, decaging is performed at a pH ranging from about 7.4 to about 7.6 in tris buffer at a temperature of about 37° C., and for a time period ranging from between about 4 minutes to about 32 minutes. It is believed that each decaging enzyme will have its own optimal conditions (buffers, salts, cofactors, temperature) and the parameters of any decaging step may be chosen to enhance the activity of the enzyme and promote “decaging” without interfering with the specific binding of the antibody conjugates.

In some embodiments, the first stage 150 also includes contacting the sample with one or more reversible enzyme inhibitors to prevent the action of the enzyme on the caging group. In some embodiments, the one or more reversible enzyme inhibitors are added after the introduction of both the unmasking antibody conjugate and the caged hapten antibody conjugate. In the context of alkaline phosphatase (AP), these reversible enzyme inhibitors may include phosphate, phenylalanine and EDTA which are believed to be able to reduce the enzyme activity by different mechanisms.

In the second stage 160, the sample is then contacted with a first set of detection reagents specific to the native hapten of the caged hapten-antibody conjugate (i.e., the first set of detection reagents are specific to an uncaged form of the hapten of the caged hapten conjugate) (step 120). Optionally, the sample is contacted with a second set of detection reagents specific to the unmasking enzyme of the unmasking enzyme-antibody conjugate (step 130). In some embodiments, steps 120 and 130 may be performed in any order or may be performed simultaneously.

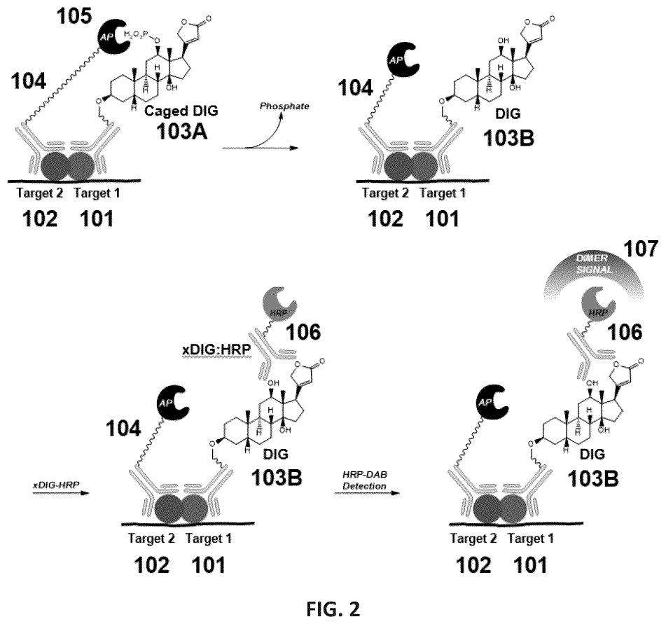

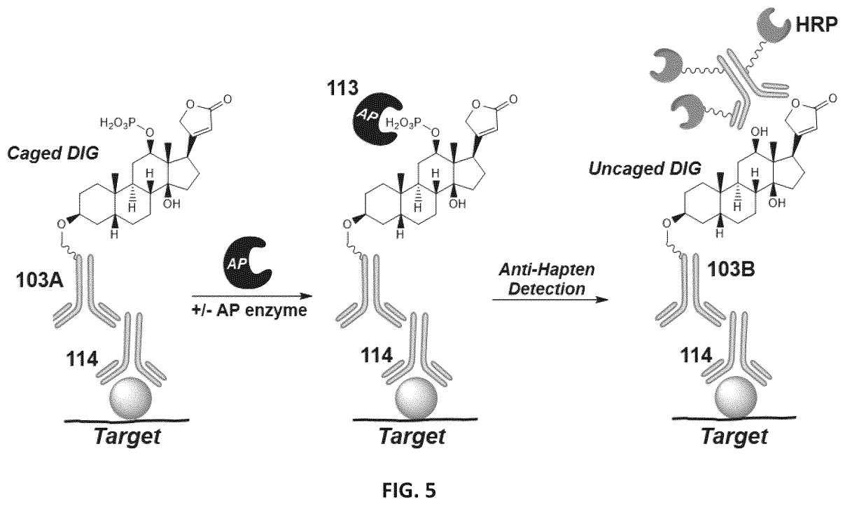

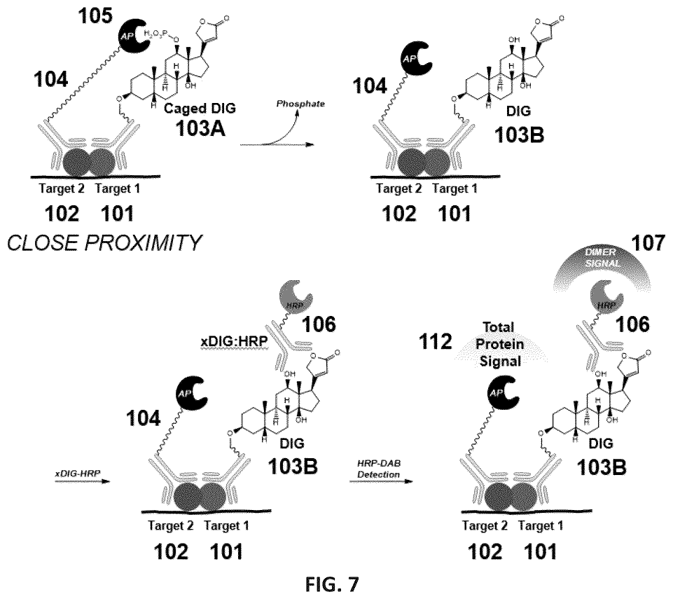

The protein proximity assays of the present disclosure are further illustrated in FIGS. 2, 3, and 5-7. For instance, FIG. 2 is a schematic illustrating the interaction between an unmasking enzyme-antibody conjugate comprising an alkaline phosphatase (bound to Target 2) and a caged hapten-antibody conjugate (bound to Target 1), where the unmasking enzyme (e.g. alkaline phosphatase) of the unmasking enzyme-antibody conjugate reacts with an enzyme substrate portion (e.g. a phosphate group or derivative thereof) of the caged hapten-antibody conjugate (by virtue of the proximity of Target 1 and Target 2 to each other) to provide the respective unmasked hapten, which may be detected. Likewise, FIG. 7 is a schematic illustrating multiplex detection of both proteins (Target 1 and Target 2) in close proximity and total protein (Target 2).

With reference to FIGS. 2 and 7, if a first target 101 is in sufficient proximity to a second target 102, the caged hapten-antibody conjugate 103A will be provided in sufficiently close proximity (the proximity being labeled 105) to the unmasking enzyme-antibody conjugate 104 such that the unmasking enzyme of the unmasking enzyme-antibody conjugate 104 may react with the enzyme substrate of the caged hapten-antibody conjugate 103A. This, in turn, results in the formation of a first target unmasked hapten-antibody conjugate complex (103B). As illustrated in FIGS. 2, 5, and 7, the first target unmasked hapten-antibody conjugate complex (103B) is able to bind or be recognized by other specific binding entities (e.g., a secondary antibody 106).

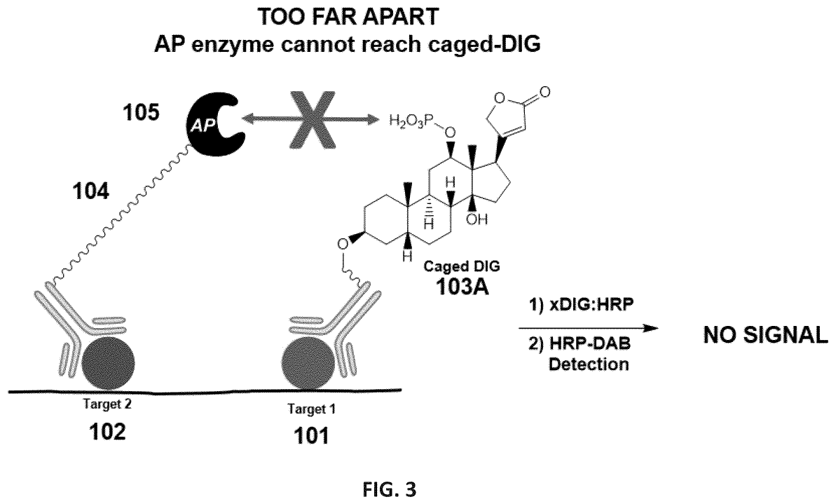

On the other hand, and as illustrated in FIG. 3, if a first target 101 is not in sufficient proximity to a second target 102, the caged hapten-antibody conjugate 103A will not be provided in proximity (the proximity being labeled 108) to the unmasking enzyme-antibody conjugate 104. In this instance, the unmasking enzyme will not be reactive with the enzyme substrate of the caged hapten-antibody conjugate 103A, and thus the caged hapten will remain in a masked or protected state, i.e., it is not capable of binding or being recognized by other specific binding entities.

Referring again to FIGS. 2, 5, and 7, following the introduction of the antibody conjugates and any decaging steps, the sample is then contacted (step 120) with first detection reagents (106), the first detection reagents being specific to the unmasked hapten of the first target unmasked hapten-antibody conjugate complex (103B). In some embodiments, the first detection reagents include a secondary antibody (106) specific for the unmasked hapten (103B), namely an anti-unmasked hapten antibody. In some embodiments, the anti-unmasked hapten antibody (106) is conjugated to a detectable moiety (e.g., in FIGS. 2 and 7, the detectable moiety is a HRP enzyme, where the HRP enzyme acts upon a substrate, such as a silver chromogenic substrate. In some embodiments, the first detection reagents (106) will only bind if the native or unmasked hapten (103B) of the first target unmasked hapten-antibody conjugate complex is unmasked by the unmasking enzyme of the unmasking enzyme-antibody conjugate (104). Thus, signal (107) from the detectable moiety of the first detection reagents (106) will only be able to be detected at step 140 if the first and second targets (101 and 102), and, hence, the antibody conjugates (103A and 104), are in close proximity to each other. Here, detected signal (107) is representative of a protein dimer or proteins/targets in close proximity (compare to FIG. 3 where the targets were not in sufficient close proximity to each other).

In some embodiments, an amplification step may be carried out to increase detectable signal. For example, amplification components may be introduced to further label the unmasked hapten of the first target unmasked hapten-antibody conjugate with additional reporter moieties, e.g., additional haptens or other “detectable moieties.” By way of example, an anti-unmasked hapten antibody conjugated to an amplification hapten (or, in other embodiments, conjugated to an enzyme) may be introduced to label the unmasked hapten of the first target unmasked hapten-antibody conjugate with a plurality of amplification haptens. Subsequently, anti-amplification hapten antibodies, each conjugated to a detectable moiety, may be introduced. In some embodiments, the anti-amplification hapten antibodies are conjugated to an enzyme, where the enzyme acts upon an introduced substrate to produce a signal (e.g., a chromogenic substrate or a fluorescent substrate to produce a visual signal). TSA and QM conjugates, each described herein, may be used in any amplification step. In some examples, signal amplification is carried out using OPTIVIEW Amplification Kit (Ventana Medical Systems, Inc., Tucson, Ariz., Catalog No. 760-099).

Multiplex Detection

In some embodiments, the unmasking enzyme of the unmasking enzyme-antibody conjugate may serve two functions, namely (i) to unmask or reveal a caged hapten; and (ii) to react with another substrate (e.g., a chromogenic substrate or a fluorescent substrate) such that a signal independent from that generated by the unmasked hapten (i.e., the unmasked hapten-antibody conjugate complex) may be detected. Accordingly, the presently disclosed system allows for the proximity between two proteins to be visualized within the context of the total protein stain for one of the proteins. Without wishing to be bound by any particular theory, it is believed that the ability to multiplex proximity detection within the context of another protein stain is a feature that allows for the possibility of having a speedy, guided slide read (i.e., only looking for proximity signal within the total protein) or the ability to quantitate the percentage of protein that is interacting with another (a method of scoring the proximity assay).

Referring again to FIGS. 2, 4, and 7 following the introduction of the first detection reagents (106), second detection reagents including a second detectable moiety ay optionally be introduced to the sample at step 130 such that total protein may be detected. In some embodiments, the second detectable moiety provides signals (112) different from that of the first detectable moiety (107). In some embodiments, the second detectable moiety comprises a substrate for the unmasking enzyme, e.g., a chromogenic substrate that provides yellow signals (109). In other embodiments, the second detectable moiety comprises a signaling conjugate.

In some embodiments, the biological samples are pre-treated with an enzyme inactivation composition to substantially or completely inactivate endogenous peroxidase activity. For example, some cells or tissues contain endogenous peroxidase. Using an HRP conjugated antibody may result in high, non-specific background staining. This non-specific background can be reduced by pre-treatment of the sample with an enzyme inactivation composition as disclosed herein. In some embodiments, the samples are pre-treated with hydrogen peroxide only (about 1% to about 3% by weight of an appropriate pre-treatment solution) to reduce endogenous peroxidase activity. Once the endogenous peroxidase activity has been reduced or inactivated, detection kits may be added, followed by inactivation of the enzymes present in the detection kits, as provided above. The disclosed enzyme inactivation composition and methods can also be used as a method to inactivate endogenous enzyme peroxidase activity. Additional inactivation compositions are described in U.S. Publication No. 2018/0120202, the disclosure of which is hereby incorporated by reference herein in its entirety.

In some embodiments if the specimen is a sample embedded in paraffin, the sample can be deparaffinized using appropriate deparaffinizing fluid(s). After a waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

Detection of Caged Hapten Antibody Conjugates

In some embodiments, detection reagents are utilized to enable detection of any of the caged hapten conjugates described herein or a complex of a caged hapten conjugate and a target, such as a target within a sample. As described herein, in some embodiments the detection reagents employed are specific to an unmasked or a native hapten corresponding to the caged hapten of any caged hapten-conjugate. For example, if the caged hapten-conjugate is a phosphorylated DIG, then detection reagents would be utilized to enable detection of DIG, which is the unmasked or native hapten corresponding to the phosphorylated DIG. Detection reagents may also include components designed to increase signal, e.g., signal amplification components or signal amplification kits.

In some embodiments, the detection reagents specific to the unmasked hapten are secondary antibodies specific to the unmasked hapten of the caged hapten conjugate, i.e., anti-unmasked hapten antibodies, and are themselves conjugated to a detectable moiety. A “detectable moiety” is a molecule or material that can produce a detectable (such as visually, electronically, or otherwise) signal that indicates the presence (i.e., qualitative analysis) and/or concentration (i.e., quantitative analysis) of the caged hapten-antibody conjugate and/or unmasking enzyme-antibody conjugate in a sample. A detectable signal can be generated by any known or yet to be discovered mechanism including absorption, emission and/or scattering of a photon (including radio frequency, microwave frequency, infrared frequency, visible frequency, and ultra-violet frequency photons).

In some embodiments, the detectable moiety of the anti-unmasked hapten antibody includes chromogenic, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected through antibody-hapten binding interactions using additional detectably labeled antibody conjugates, and paramagnetic and magnetic molecules or materials. Of course, the detectable moieties can themselves also be detected indirectly, e.g., if the detectable moiety is a hapten, then yet another antibody specific to that detectable moiety may be utilized in the detection of the detectable moiety, as known to those of ordinary skill in the art.

In some embodiments, the anti-unmasked hapten antibody includes a detectable moiety selected from the group consisting of Cascade Blue acetyl azide; Dapoxylsulfonic acid/carboxylic acid DY-405; Alexa Fluor 405 Cascade Yellow pyridyloxazole succinimidyl ester (PyMPO); Pacific Blue DY-415; 7-hydroxycoumarin-3-carboxylic acid DYQ-425; 6-FAM phosphoramidite; Lucifer Yellow; Alexa Fluor 430 Dabcyl NBD chloride/fluoride; QSY 35 DY-485XL; Cy2 DY-490; Oregon Green 488 Alexa Fluor 488 BODIPY 493/503 C3 DY-480XL; BODIPY FL C3 BODIPY FL C5 BODIPY FL-X DYQ-505; Oregon Green 514 DY-510XL; DY-481XL; 6-carboxy-4′,5′-dichloro-2′,7′-dimethoxyfluorescein succinimidyl ester (JOE); DY-520XL; DY-521XL; BODIPY R6G C3 erythrosin isothiocyanate; 5-carboxy-2′,4′,5′,7′-tetrabromosulfonefluorescein Alexa Fluor 532 6-carboxy-2′,4,4′,5′7,7′-hexachlorofluorescein succinimidyl ester (HEX); BODIPY 530/550 C3 DY-530; BODIPY TMR-X DY-555; DYQ-1; DY-556; Cy3 DY-547; DY-549; DY-550; Alexa Fluor 555 Alexa Fluor 546 DY-548; BODIPY 558/568 C3 Rhodamine red-X QSY 7 BODIPY 564/570 C3 BODIPY 576/589 C3 carboxy-X-rhodamine (ROX); Alexa Fluor 568 DY-590; BODIPY 581/591 C3 DY-591; BODIPY TR-X Alexa Fluor 594 DY-594; carboxynaphthofluorescein DY-605; DY-610; Alexa Fluor 610 DY-615; BODIPY 630/650-X erioglaucine; Alexa Fluor 633 Alexa Fluor 635 succinimidyl ester; DY-634; DY-630; DY-631; DY-632; DY-633; DYQ-2; DY-636; BODIPY 650/665-X DY-635; Cy5 Alexa Fluor 647 DY-647; DY-648; DY-650; DY-654; DY-652; DY-649; DY-651; DYQ-660; DYQ-661; Alexa Fluor 660 Cy5.5 DY-677; DY-675; DY-676; DY-678; Alexa Fluor 680 DY-679; DY-680; DY-682; DY-681; DYQ-3; DYQ-700; Alexa Fluor 700 DY-703; DY-701; DY-704; DY-700; DY-730; DY-731; DY-732; DY-734; DY-750; Cy7 DY-749; DYQ-4; and Cy7.5.

Fluorophores belong to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11^{th} Edition. In other embodiments, the fluorophore is selected from xanthene derivatives, cyanine derivatives, squaraine derivatives, naphthalene derivatives, coumarin derivatives, oxadiazole derivatives, anthracene derivatives, pyrene derivatives, oxazine derivatives, acridine derivatives, arylmethine derivatives, and tetrapyrrole derivatives. In other embodiments, the fluorescent moiety is selected from a CF dye (available from Biotium), DRAQ and CyTRAK probes (available from BioStatus), BODIPY (available from Invitrogen), Alexa Fluor (available from Invitrogen), DyLight Fluor (e.g. DyLight 649) (available from Thermo Scientific, Pierce), Atto and Tracy (available from Sigma Aldrich), FluoProbes (available from Interchim), Abberior Dyes (available from Abberior), DY and MegaStokes Dyes (available from Dyomics), Sulfo Cy dyes (available from Cyandye), HiLyte Fluor (available from AnaSpec), Seta, SeTau and Square Dyes (available from SETA BioMedicals), Quasar and Cal Fluor dyes (available from Biosearch Technologies), SureLight Dyes (available from APC, RPEPerCP, Phycobilisomes) (Columbia Biosciences), and APC, APCXL, RPE, BPE (available from Phyco-Biotech, Greensea, Prozyme, Flogen).

In other embodiments, the anti-unmasked hapten antibody is conjugated to an enzyme. In these embodiments, the final proximity signal can be generated with any enzyme conjugated to the relevant anti-unmasked hapten antibody, with the exception of the enzyme that is used for unmasking (e.g., an unmasking enzyme of an unmasking enzyme-antibody conjugate, described further herein). In some embodiments, suitable enzymes include, but are not limited to, horseradish peroxidase, alkaline phosphatase, acid phosphatase, glucose oxidase, neuramindase, β-galactosidase, β-glucuronidase or β-lactamase. In other embodiments, enzymes include oxidoreductases or peroxidases (e.g., HRP). In these embodiments, the enzyme conjugated to the anti-unmasked hapten antibody catalyzes conversion of a chromogenic substrate, a covalent hapten, a covalent fluorophore, non-covalent chromogens, and non-covalent fluorophores to a reactive moiety which labels a sample proximal to or directly on the target.

Particular non-limiting examples of chromogenic compounds/substrates include diaminobenzidine (DAB), 4-nitrophenylphospate (pNPP), fast red, bromochloroindolyl phosphate (BCIP), nitro blue tetrazolium (NBT), BCIP/NBT, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazole (AEC), fuchsin, iodonitrotetrazolium (INT), tetrazolium blue, and tetrazolium violet. DAB, which is oxidized in the presence of peroxidase and hydrogen peroxide, results in the deposition of a brown, alcohol-insoluble precipitate at the site of enzymatic activity.

In some embodiments, the chromogenic substrates are signaling conjugates which comprise a latent reactive moiety and a chromogenic moiety. In some embodiments, the latent reactive moiety of the signaling conjugate is configured to undergo catalytic activation to form a reactive species that can covalently bond with the sample or to other detection components. The catalytic activation is driven by one or more enzymes (e.g., oxidoreductase enzymes and peroxidase enzymes, like horseradish peroxidase) and results in the formation of a reactive species. These reactive species are capable of reacting with the chromogenic moiety proximal to their generation, i.e., near the enzyme. Specific examples of signaling conjugates are disclosed in US Patent Publication No. 2013/0260379, the disclosure of which is hereby incorporated by reference herein in its entirety.

Other substrates include those set forth in U.S. Pat. No. 5,583,001, U.S. application publication No. 2012/0171668, and PCT/EP2015/0533556, the disclosures of which are hereby incorporate by reference herein in their entireties. Suitable chromogenic substrates or fluorescent substrates coupled to TSA or QM conjugates, as noted in the above incorporated references, include N,N′-biscarboxypentyl-5,5′-disulfonato-indo-dicarbocyanine (Cy5), 4-(dimethylamino) azobenzene-4′-sulfonamide (Dabsyl), tetramethylrhodamine (Tamra), and Rhodamine 110 (Rhodamine).

In some embodiments, the chromogenic substrates, fluorescent substrates, or signaling conjugates are selected such that peak detectable wavelengths of any chromogenic moiety do not overlap with each other and are readily detectable by a pathologist or an optical detector (e.g., a scanner). In some embodiments, the chromogenic moieties are selected such that the peak wavelengths of the different chromogenic moieties are separated by at least about 50 nm. In other embodiments, the chromogenic moieties are selected such that the peak wavelengths of the different chromogenic moieties are separated by at least about 70 nm. In yet other embodiments, the chromogenic moieties are selected such that the peak wavelengths of the different chromogenic moieties are separated by at least about 100 nm. Examples of suitable detectable moieties having a coumarin core are described in U.S. Pat. No. 10,041,950, the disclosure of which is hereby incorporated by reference herein in its entirety. Other suitable detectable moieties are disclosed in U.S. Provisional patent Application No. 63/071,518, the disclosure of which is hereby incorporated by reference herein in its entirety.

In yet further embodiments, the chromogenic moieties are selected such that the chromogenic moieties, when introduced to the tissue specimen, provide for different colors (e.g., yellow, blue, magenta). In some embodiments, the chromogenic moieties are selected such that they provide a good contrast between each other, e.g., a separation of colors that are optically recognizable. In some embodiments, the chromogenic moieties are selected such that when placed in close proximity of each other provide for a signal or color that is different than the signals or colors of either of the chromogenic moieties when observed alone.

Kits

In some embodiments, the caged hapten conjugates of the present disclosure may be utilized as part of a “detection kit.” In general, any detection kit may include one or more caged hapten conjugates and detection reagents for detecting the one or more caged hapten conjugates. In some embodiments, the kit comprises a caged hapten conjugate of any of Formulas (IVA), (IVB), or (VA)-(VF).

The detection kits may include a first composition comprising a caged hapten conjugate and a second composition comprising detection reagents specific to the first composition, such that the caged hapten conjugate may be detected via the detection kit. In some embodiments, the detection kit includes a plurality of caged hapten conjugates (such as mixed together in a buffer), where the detection kit also includes detection reagents specific for each of the plurality of caged hapten conjugates.

Of course, any kit may include other agents, including buffers; counterstaining agents; enzyme inactivation compositions; deparaffinization solutions, etc. as needed for manual or automated target detection. The kit may also include instructions for using any of the components of the kit, including methods of applying the kit components to a tissue sample to effect detection of one or more targets therein.

Automation

The assays and methods of the present disclosure may be automated and may be combined with a specimen processing apparatus. The specimen processing apparatus can be an automated apparatus, such as the BENCHMARK Ultra instrument and DISCOVERY Ultra instrument sold by Ventana Medical Systems, Inc. Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. Alternatively, specimens can be manually processed.

The specimen processing apparatus can apply fixatives to the specimen. Fixatives can include cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation).

If the specimen is a sample embedded in paraffin, the sample can be deparaffinized with the specimen processing apparatus using appropriate deparaffinizing fluid(s). After the waste remover removes the deparaffinizing fluid(s), any number of substances can be successively applied to the specimen. The substances can be for pretreatment (e.g., protein-crosslinking, expose nucleic acids, etc.), denaturation, hybridization, washing (e.g., stringency wash), detection (e.g., link a visual or marker molecule to a probe), amplifying (e.g., amplifying proteins, genes, etc.), counterstaining, coverslipping, or the like.

The specimen processing apparatus can apply a wide range of substances to the specimen. The substances include, without limitation, stains, probes, reagents, rinses, and/or conditioners. The substances can be fluids (e.g., gases, liquids, or gas/liquid mixtures), or the like. The fluids can be solvents (e.g., polar solvents, non-polar solvents, etc.), solutions (e.g., aqueous solutions or other types of solutions), or the like. Reagents can include, without limitation, stains, wetting agents, antibodies (e.g., monoclonal antibodies, polyclonal antibodies, etc.), antigen recovering fluids (e.g., aqueous- or non-aqueous-based antigen retrieval solutions, antigen recovering buffers, etc.), or the like. Probes can be an isolated nucleic acid or an isolated synthetic oligonucleotide, attached to a detectable label. Labels can include radioactive isotopes, enzyme substrates, co-factors, ligands, chemiluminescent or fluorescent agents, haptens, and enzymes.

After the specimens are processed, a user can transport specimen-bearing slides to the imaging apparatus. The imaging apparatus used here is a brightfield imager slide scanner. One brightfield imager is the iScan Coreo™ brightfield scanner sold by Ventana Medical Systems, Inc. In automated embodiments, the imaging apparatus is a digital pathology device as disclosed in International Patent Application No.: PCT/US2010/002772 (Patent Publication No.: WO/2011/049608) entitled IMAGING SYSTEM AND TECHNIQUES or disclosed in U.S. Patent Application Publication No. 2014/0178169, filed on Feb. 3, 2014, entitled IMAGING SYSTEMS, CASSETTES, AND METHODS OF USING THE SAME. International Patent Application No. PCT/US2010/002772 and U.S. Patent Application Publication No. 2014/0178169 are incorporated by reference in their entities. In other embodiments, the imaging apparatus includes a digital camera coupled to a microscope.

Counterstaining

Counterstaining is a method of post-treating the samples after they have already been stained with agents to detect one or more targets, such that their structures can be more readily visualized under a microscope. For example, a counterstain is optionally used prior to coverslipping to render the immunohistochemical stain more distinct. Counterstains differ in color from a primary stain. Numerous counterstains are well known, such as hematoxylin, eosin, methyl green, methylene blue, Giemsa, Alcian blue, and Nuclear Fast Red. DAPI (4′,6-diamidino-2-phenylindole) is a fluorescent stain that may be used.

In some examples, more than one stain can be mixed together to produce the counterstain. This provides flexibility and the ability to choose stains. For example, a first stain, can be selected for the mixture that has a particular attribute, but yet does not have a different desired attribute. A second stain can be added to the mixture that displays the missing desired attribute. For example, toluidine blue, DAPI, and pontamine sky blue can be mixed together to form a counterstain.

Detection and/or Imaging

Certain aspects, or all, of the disclosed embodiments can be automated, and facilitated by computer analysis and/or image analysis system. In some applications, precise color or fluorescence ratios are measured. In some embodiments, light microscopy is utilized for image analysis. Certain disclosed embodiments involve acquiring digital images. This can be done by coupling a digital camera to a microscope. Digital images obtained of stained samples are analyzed using image analysis software. Color or fluorescence can be measured in several different ways. For example, color can be measured as red, blue, and green values; hue, saturation, and intensity values; and/or by measuring a specific wavelength or range of wavelengths using a spectral imaging camera. The samples also can be evaluated qualitatively and semi-quantitatively. Qualitative assessment includes assessing the staining intensity, identifying the positively-staining cells and the intracellular compartments involved in staining, and evaluating the overall sample or slide quality. Separate evaluations are performed on the test samples and this analysis can include a comparison to known average values to determine if the samples represent an abnormal state.

Suitable detection methods are described in in PCT Application No. WO/2014/143155, the disclosure of which is hereby incorporated by reference herein in its entirety. In some embodiments, a suitable detection system comprises an imaging apparatus, one or more lenses, and a display in communication with the imaging apparatus. The imaging apparatus includes means for sequentially emitting energy and means for capturing an image/video. In some embodiments, the means for capturing is positioned to capture specimen images, each corresponding to the specimen being exposed to energy. In some embodiments, the means for capturing can include one or more cameras positioned on a front side and/or a backside of the microscope slide carrying the biological sample. The display means, in some embodiments, includes a monitor or a screen. In some embodiments, the means for sequentially emitting energy includes multiple energy emitters. Each energy emitter can include one or more IR energy emitters, UV energy emitters, LED light emitters, combinations thereof, or other types of energy emitting devices. The imaging system can further include means for producing contrast enhanced color image data based on the specimen images captured by the means for capturing. The displaying means displays the specimen based on the contrast enhanced color image data.

Samples and Targets

Samples include biological components and generally are suspected of including one or more target molecules of interest. Target molecules can be on the surface of cells and the cells can be in a suspension, or in a tissue section. Target molecules can also be intracellular and detected upon cell lysis or penetration of the cell by a probe. One of ordinary skill in the art will appreciate that the method of detecting target molecules in a sample will vary depending upon the type of sample and probe being used. Methods of collecting and preparing samples are known in the art.

Samples for use in the embodiments of the method and with the composition disclosed herein, such as a tissue or other biological sample, can be prepared using any method known in the art by of one of ordinary skill. The samples can be obtained from a subject for routine screening or from a subject that is suspected of having a disorder, such as a genetic abnormality, infection, or a neoplasia. The described embodiments of the disclosed method can also be applied to samples that do not have genetic abnormalities, diseases, disorders, etc., referred to as “normal” samples. Such normal samples are useful, among other things, as controls for comparison to other samples. The samples can be analyzed for many different purposes. For example, the samples can be used in a scientific study or for the diagnosis of a suspected malady, or as prognostic indicators for treatment success, survival, etc.

Samples can include multiple targets that can be specifically bound by a probe or reporter molecule. The targets can be nucleic acid sequences or proteins. Throughout this disclosure when reference is made to a target protein it is understood that the nucleic acid sequences associated with that protein can also be used as a target. In some examples, the target is a protein or nucleic acid molecule from a pathogen, such as a virus, bacteria, or intracellular parasite, such as from a viral genome. For example, a target protein may be produced from a target nucleic acid sequence associated with (e.g., correlated with, causally implicated in, etc.) a disease.

A target nucleic acid sequence can vary substantially in size. Without limitation, the nucleic acid sequence can have a variable number of nucleic acid residues. For example, a target nucleic acid sequence can have at least about 10 nucleic acid residues, or at least about 20, 30, 50, 100, 150, 500, 1000 residues. Similarly, a target polypeptide can vary substantially in size. Without limitation, the target polypeptide will include at least one epitope that binds to a peptide specific antibody, or fragment thereof. In some embodiments that polypeptide can include at least two epitopes that bind to a peptide specific antibody, or fragment thereof.

In specific, non-limiting examples, a target protein is produced by a target nucleic acid sequence (e.g., genomic target nucleic acid sequence) associated with a neoplasm (for example, a cancer). Numerous chromosome abnormalities (including translocations and other rearrangements, amplification, or deletion) have been identified in neoplastic cells, especially in cancer cells, such as B cell and T cell leukemias, lymphomas, breast cancer, colon cancer, neurological cancers and the like. Therefore, in some examples, at least a portion of the target molecule is produced by a nucleic acid sequence (e.g., genomic target nucleic acid sequence) amplified or deleted in at least a subset of cells in a sample.

Oncogenes are known to be responsible for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes FPC nucleic acid molecules generated from a target nucleic acid sequence that extends distally from the SYT gene, and the second probe includes FPC nucleic acid generated from a target nucleic acid sequence that extends 3′ or proximal to the SYT gene. When probes corresponding to these target nucleic acid sequences (e.g., genomic target nucleic acid sequences) are used in an in-situ hybridization procedure, normal cells, which lack a t(18q11.2) in the SYT gene region, exhibit two fusions (generated by the two labels in close proximity) signals, reflecting the two intact copies of SYT. Abnormal cells with a t(18q11.2) exhibit a single fusion signal.

In other examples, a target protein produced from a nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1) are characteristic molecular features of certain types of solid tumors of the central nervous system.

The aforementioned examples are provided solely for purpose of illustration and are not intended to be limiting. Numerous other cytogenetic abnormalities that correlate with neoplastic transformation and/or growth are known to those of ordinary skill in the art. Target proteins that are produced by nucleic acid sequences (e.g., genomic target nucleic acid sequences), which have been correlated with neoplastic transformation and which are useful in the disclosed methods, also include the EGFR gene (7p12; e.g., GENBANK™ Accession No. NC 000007, nucleotides 55054219-55242525), the C-MYC gene (8q24.21; e.g., GENBANK™ Accession No. NC 000008, nucleotides 128817498-128822856), D5S271 (5p15.2), lipoprotein lipase (LPL) gene (8p22; e.g., GENBANK™ Accession No. NC-000008, nucleotides 19841058-19869049), RB1 (13q14; e.g., GENBANK™ Accession No. NC 000013, nucleotides 47775912-47954023), p53 (17p13.1; e.g., GENBANK™ Accession No. NC 000017, complement, nucleotides 7512464-7531642)), N-MYC (2p24; e.g., GENBANK™ Accession No. NC-000002, complement, nucleotides 151835231-151854620), CHOP (12q13; e.g., GENBANK™ Accession No. NC 000012, complement, nucleotides 56196638-56200567), FUS (16p11.2; e.g., GENBANK™ Accession No. NC 000016, nucleotides 31098954-31110601), FKHR (13p14; e.g., GENBANK™ Accession No. NC-000013, complement, nucleotides 40027817-40138734), as well as, for example: ALK (2p23; e.g., GENBANK™ Accession No. NC-000002, complement, nucleotides 29269144-29997936), Ig heavy chain, CCND1 (11q13; e.g., GENBANK™ Accession No. NC-000011, nucleotides 69165054.69178423), BCL2 (18q21.3; e.g., GENBANK™ Accession No. NC-000018, complement, nucleotides 58941559-59137593), BCL6 (3q27; e.g., GENBANK™ Accession No. NC-000003, complement, nucleotides 188921859-188946169), MALF1, AP1 (1p32-p31; e.g., GENBANK™ Accession No. NC 000001, complement, nucleotides 59019051-59022373), TOP2A (17q21-q22; e.g., GENBANK™ Accession No. NC 000017, complement, nucleotides 35798321-35827695), TMPRSS (21q22.3; e.g., GENBANK™ Accession No. NC-000021, complement, nucleotides 41758351-41801948), ERG (21q22.3; e.g., GENBANK™ Accession No. NC 000021, complement, nucleotides 38675671-38955488); ETV1 (7p21.3; e.g., GENBANK™ Accession No. NC-000007, complement, nucleotides 13897379-13995289), EWS (22q12.2; e.g., GENBANK™ Accession No. NC 000022, nucleotides 27994271-28026505); FLI1 (11q24.1-q24.3; e.g., GENBANK™ Accession No. NC-000011, nucleotides 128069199-128187521), PAX3 (2q35-q37; e.g., GENBANK™ Accession No. NC-000002, complement, nucleotides 222772851-222871944), PAX7 (1p36.2-p36.12; e.g., GENBANK™ Accession No. NC 000001, nucleotides 18830087-18935219), PTEN (10q23.3; e.g., GENBANK™ Accession No. NC-000010, nucleotides 89613175-89716382), AKT2 (19q13.1-q13.2; e.g., GENBANK™ Accession No. NC 000019, complement, nucleotides 45431556-45483036), MYCL1 (1p34.2; e.g., GENBANK™ Accession No. NC 000001, complement, nucleotides 40133685-40140274), REL (2p13-p12; e.g., GENBANK™ Accession No. NC-000002, nucleotides 60962256-61003682) and CSF1R (5q33-q35; e.g., GENBANK™ Accession No. NC-000005, complement, nucleotides 149413051-149473128).

In other examples, a target protein is selected from a virus or other microorganism associated with a disease or condition. Detection of the virus- or microorganism-derived target nucleic acid sequence (e.g., genomic target nucleic acid sequence) in a cell or tissue sample is indicative of the presence of the organism. For example, the target peptide, polypeptide, or protein can be selected from the genome of an oncogenic or pathogenic virus, a bacterium, or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some examples, the target protein is produced from a nucleic acid sequence (e.g., genomic target nucleic acid sequence) from a viral genome. Exemplary viruses and corresponding genomic sequences (GENBANK™ RefSeq Accession No. in parentheses) include human adenovirus A (NC-001460), human adenovirus B (NC-004001), human adenovirus C(NC 001405), human adenovirus D (NC-002067), human adenovirus E (NC-003266), human adenovirus F (NC-001454), human astrovirus (NC-001943), human BK polyomavirus (V01109; GI:60851) human bocavirus (NC-007455), human coronavirus 229E (NC-002645), human coronavirus HKU1 (NC-006577), human coronavirus NL63 (NC-005831), human coronavirus OC43 (NC-005147), human enterovirus A (NC-001612), human enterovirus B (NC-001472), human enterovirus C(NC-001428), human enterovirus D (NC-001430), human erythrovirus V9 (NC-004295), human foamy virus (NC-001736), human herpesvirus 1 (Herpes simplex virus type 1) (NC-001806), human herpesvirus 2 (Herpes simplex virus type 2) (NC 001798), human herpesvirus 3 (Varicella zoster virus) (NC-001348), human herpesvirus 4 type 1 (Epstein-Barr virus type 1) (NC-007605), human herpesvirus 4 type 2 (Epstein-Barr virus type 2) (NC-009334), human herpesvirus 5 strain AD 169 (NC-001347), human herpesvirus 5 strain Merlin Strain (NC-006273), human herpesvirus 6A (NC-001664), human herpesvirus 6B (NC-000898), human herpesvirus 7 (NC-001716), human herpesvirus 8 type M (NC 003409), human herpesvirus 8 type P (NC-009333), human immunodeficiency virus 1 (NC 001802), human immunodeficiency virus 2 (NC-001722), human metapneumovirus (NC 004148), human papillomavirus-1 (NC-001356), human papillomavirus-18 (NC-001357), human papillomavirus-2 (NC-001352), human papillomavirus-54 (NC-001676), human papillomavirus-61 (NC-001694), human papillomavirus-cand90 (NC-004104), human papillomavirus RTRX7 (NC-004761), human papillomavirus type 10 (NC-001576), human papillomavirus type 101 (NC-008189), human papillomavirus type 103 (NC-008188), human papillomavirus type 107 (NC-009239), human papillomavirus type 16 (NC-001526), human papillomavirus type 24 (NC-001683), human papillomavirus type 26 (NC-001583), human papillomavirus type 32 (NC-001586), human papillomavirus type 34 (NC-001587), human papillomavirus type 4 (NC-001457), human papillomavirus type 41 (NC-001354), human papillomavirus type 48 (NC-001690), human papillomavirus type 49 (NC-001591), human papillomavirus type 5 (NC-001531), human papillomavirus type 50 (NC-001691), human papillomavirus type 53 (NC-001593), human papillomavirus type 60 (NC-001693), human papillomavirus type 63 (NC-001458), human papillomavirus type 6b (NC-001355), human papillomavirus type 7 (NC-001595), human papillomavirus type 71 (NC-002644), human papillomavirus type 9 (NC-001596), human papillomavirus type 92 (NC-004500), human papillomavirus type 96 (NC-005134), human parainfluenza virus 1 (NC-003461), human parainfluenza virus 2 (NC-003443), human parainfluenza virus 3 (NC-001796), human parechovirus (NC-001897), human parvovirus 4 (NC-007018), human parvovirus B19 (NC 000883), human respiratory syncytial virus (NC-001781), human rhinovirus A (NC-001617), human rhinovirus B (NC-001490), human spumaretrovirus (NC-001795), human T-lymphotropic virus 1 (NC-001436), human T-lymphotropic virus 2 (NC-001488).

In certain examples, the target protein is produced from a nucleic acid sequence (e.g., genomic target nucleic acid sequence) from an oncogenic virus, such as Epstein-Barr Virus (EBV) or a Human Papilloma Virus (HPV, e.g., HPV16, HPV18). In other examples, the target protein produced from a nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., SARS virus), an Adenovirus, a Polyomavirus, a Cytomegalovirus (CMV), or a Herpes Simplex Virus (HSV).

### EXAMPLES

### Example 1—Synthesis of the Compounds of the Present Disclosure

MS data was collected on a Waters Acquity QDa (ESI) running Empower 3 (Waters). Analytical HPLC was performed using Waters XBridge columns on a Waters Alliance e2695 running Empower 3 (Waters). Prep HPLC was performed with Waters SunFire columns (Prep C18 OBD 10 μm, 50 mm×250 mm) on a Waters 2535 running Empower 3 (Waters). All chemicals were purchased from commercial suppliers and used as received unless otherwise noted.

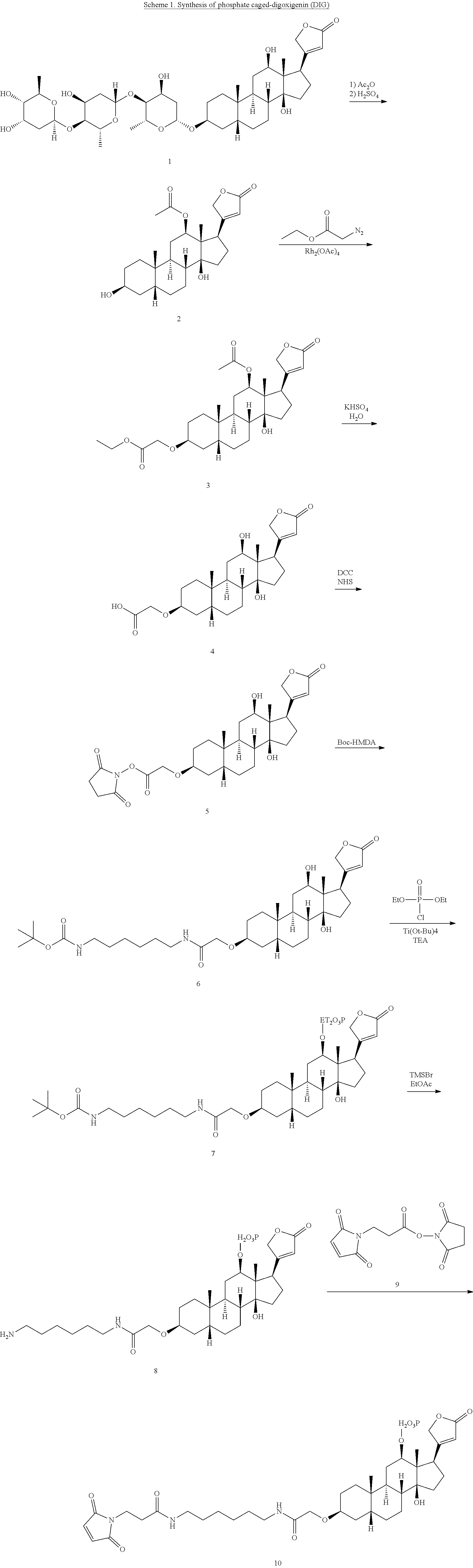

Compound 4 was prepared by acetylation and acidic hydrolysis of digoxin 1 to give 12-O-acetyldigoxigenin 2, which was converted by a reaction with ethyl diazoacetate into 3-ethyloxycarbonylmethyl ether 3, followed by its hydrolysis into digoxigenin-3-carboxymethyl ether 4 as described in the patent [U.S. Pat. No. 5,198,537]

Compound 6. To a stirred solution of digoxigenin-3-carboxymethyl ether 4 (1.0 eq) in THF (6 mL per mmol 4) was added N-hydroxysuccinimide (1.5 eq) and 1M of N,N′-dicyclohexylcarbodiimide (1.5 eq) in CH₂Cl₂. The reaction mixture was kept at room temperature for 20 h (check HPLC to confirm reaction completion), filtered and the solvents removed under reduced pressure. The residue was diluted with EtOAc. The resulting solution was filtered again, followed by washing with brine. The organic layer was dried over MgSO₄, and the solvents removed under reduced pressure to give the NHS ester 5, which was dissolved in EtOAc (6 mL per mmol 5), followed by addition of TEA (1.5 eq) and N-Boc-ethylenediamine (1.5 eq). The reaction mixture was stirred at RT for 1 h (check HPLC to confirm reaction completion), diluted with EtOAc (6 mL per mmol 5) followed by addition of 1M HCl (10 mL per mmol 5). The organic layer was separated, followed by washing with saturated NaHCO₃ and brine. The organic layer was dried over MgSO₄, and the solvents removed under reduced pressure to give compound 6, which was used without further purification. MS (ESI) m/z (M+H-Boc)⁺ calculated for C₃₁H₅₁N₂O₆⁺ 547.3, found 547.3.

Compound 7. To a solution of compound 6 (1.0 eq), Ti(O-t-Bu)₄ (0.2 eq), and TEA (3.5 eq) in CH₂Cl₂ (2 mL per mmol 6) was added diethyl chlorophosphate (2.5 eq). The reaction vessel was sealed and stirred at room temperature for 16 h (monitored by HPLC to confirm reaction was ˜30% complete). The reaction mixture was diluted with EtOAc (50 mL per mmol 6), followed by washing with 0.5M HCl (50 mL per mmol 6). The organic layer was dried over MgSO₄, and the solvents removed under reduced pressure to give an off-white foam. The reaction was repeated 2 times more, at which point the HPLC showed the reaction was ˜90% complete compared to the starting material 6. The crude oil was purified by prep RP-HPLC (0.05% TFA in H₂O:MeCN 95:5 to 5:95 over 40 min; 40 ml/min) to give diethyl phosphate 7 as an off-white foam (35% yield from 4). MS (ESI) m/z (M+H-Boc)⁺ calcd for C₃₅H₆₀N₂O₉P⁺ 683.4, found 683.5.

Compound 8. Compound 7 (1.0 eq) was dissolved in CHCl₃ (2 mL per mmol 7), followed by addition of EtOAc (0.2 eq) and TMSBr (3.3 eq). The resulting reaction mixture was stirred at RT for 18 h (monitored by HPLC to confirm reaction was >95% complete). The solvents were removed under reduced pressure, followed by addition of MeOH (6 mL per mmol 7). The solvents were again removed under reduced pressure, and the resulting residue was purified by prep RP-HPLC (0.05% TFA in H₂O:MeCN 95:5 to 5:95 over 40 min; 40 ml/min) to give phosphate 8 as a white solid (45% yield). MS (ESI) m/z (M+H)⁺ calcd for C₃₁H₅₂N₂O₉P⁺ 627.3, found 627.4.

Compound 10. Compound 8 (1.0 eq) was suspended in DMF (2 mL per mmol 8), followed by addition of triethylamine (5 eq) and finally 3-maleimidopropionic acid NHS ester 9 (1.1 eq). The reaction vessel was sealed, and the reaction mixture was vigorously stirred at RT for 4 h (check HPLC to confirm reaction completion). The reaction mixture was then diluted with MeOH and directly purified by prep RP-HPLC (0.05% TFA in H₂O:MeCN 99:1 to 5:95 over 40 min) to give compound 10 as a light-yellow solid. MS (ESI) m/z (M+H)⁺ calcd for C₃₅H₅₇N₃O₁₂P⁺ 778.4, found 778.5.

### Example 2—Antibody Conjugate Preparation

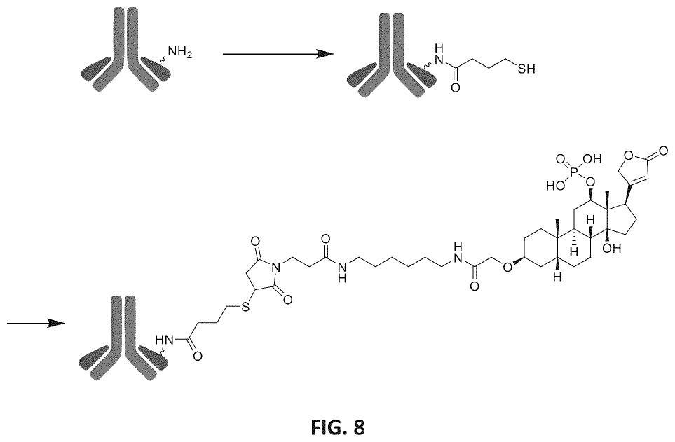

20 mg of goat-anti-rabbit IgG in 2 ml of 1×PBS (pH 7.2) was added to EDTA to provide a final concentration of 10 mM followed by 2 mg of Traut's reagent (2-iminothiolane hydrochloride). The reaction mixture was kept at room temperature for 1 h followed by size exclusion chromatography purification (AKTA, Superdex 200 10/300 GL column) with 1×PBS (pH 7.2), containing 10 mM EDTA. To the combined fractions of thiolated antibody (6 mg/ml) were added 4.2 mg of compound 10 in 0.2 ml of DMF. The reaction mixture was kept at room temperature for 3 h followed by size exclusion chromatography purification (AKTA, Superdex 200 10/300 GL column) with 1×PBS (pH 7.2) to give caged digoxigenin-modified antibody (3.7 mg/ml). FIG. 8 illustrates the conjugation of an antibody to a caged hapten of the present disclosure.

### Example 3—Stability Study

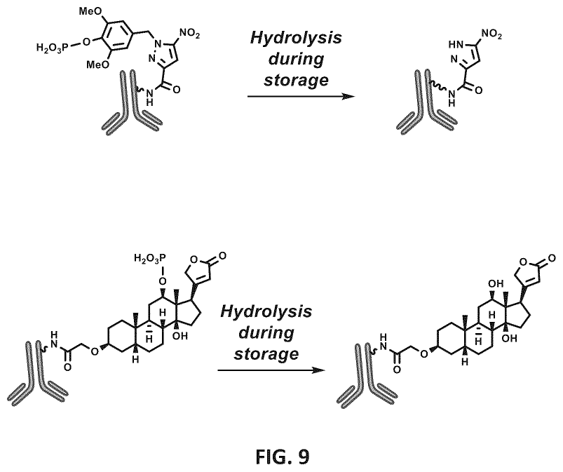

To study the hydrolytic stability of caged hapten conjugates having any one of Formulas (IVA), (IVB), or (VA)-(VF) (see FIG. 9) model compounds were subjected to a stability study at elevated temperatures.

Model compounds tested were the NP hapten with two different caging groups along with caged DIG, as set forth below:

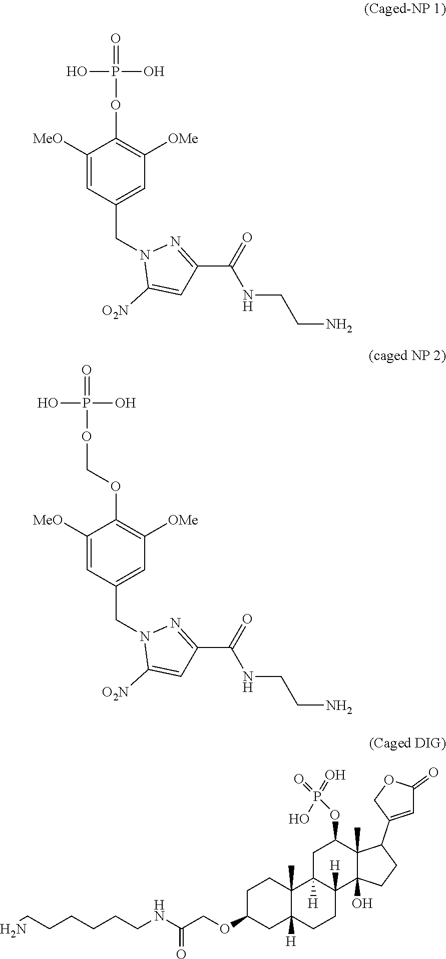

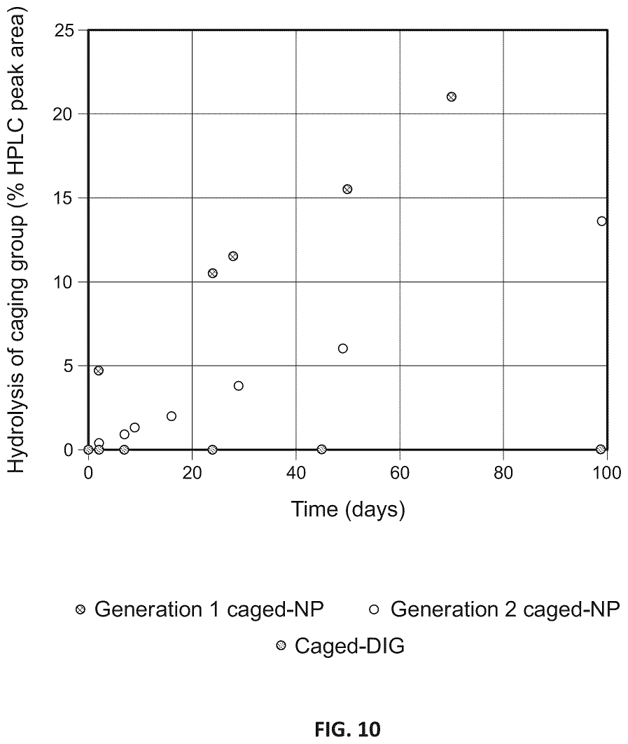

The samples of caged NP and caged DIG were stored in 100 mM PBS (pH 7.2) in a 37° C. oven. The buffer and pH were representative of storage conditions for an antibody conjugate. A temperature of 37° C. was chosen to stress the sample and accelerate hydrolytic events. Normal storage conditions or the antibody conjugate were believed to be about 4° C. Aliquots of the samples were removed at regular intervals and tested by reverse phase HPLC on a Waters XBridge column on a Waters Alliance e2695 running Empower 3 (Waters). Each sample HPLC trace was examined for evidence of decaging or other forms of decomposition. After 50 days of storage at 37° C. the generation 1 caged-NP showed ˜15.5% hydrolysis, the generation 2 caged-NP had ˜6% hydrolysis, whereas the caged DIG had <0.5% hydrolysis (see FIG. 10). The caged DIG testing was monitored out to 120 days and <0.5% hydrolysis was still observed at this point. It was concluded that the caged haptens of the present disclosure exhibited excellent hydrolytic stability.

### Example 4—Immunohistochemistry

General immunohistochemistry (IHC) protocols.

All IHC staining experiments were carried out on a VENTANA DISCOVERY® Ultra automated tissue staining platform. The reagents used in these protocols were from Roche Tissue Diagnostics (Tucson, AZ, USA; “RTD”) unless otherwise specified.

Proximity IHC General Procedure

All formalin fixed, paraffin embedded (FFPE) tissue and cell line samples were mounted on Superfrost Plus glass slides (Fisher Scientific, #12-550-15). These were deparaffinized using EZ Prep (RTD, #950-101). Heat induced epitope retrieval (HIER), or antigen retrieval (AR) was performed with Cell Conditioning 1 (CC1, RTD, #950-124). The general steps after deparaffinization and AR were: (1) inactivation of endogenous peroxidases with Inhibitor CM (RTD, 760-4307); (2) co-incubation with the primary antibodies (about 37° C., time ranging from about 8 to about 32 minutes depending on the antibodies); (3) incubation with a goat-anti-mouse secondary antibody conjugated to alkaline phosphatase (AP); (4) incubation with a goat-anti-rabbit secondary antibody conjugated to caged haptens; (5) incubation with a mouse-anti-hapten HRP conjugate; (6) signal amplification with tyramide-HQ and H₂O₂(RTD, #760-052); (7) incubation with a mouse-anti-HQ HRP conjugate (RTD, #760-4602); (8) detection with 3,3′-diaminobenzidine (DAB), hydrogen peroxide (H₂O₂), and toning with copper; (9) counterstaining with Hematoxylin II (RTD, #790-2208) and Bluing (RTD, #760-2037) to stain the nuclei; (10) dehydration with gradient alcohols and xylenes, followed by coverslipping. The slides were washed with Reaction Buffer (RTD, #950-300) between each of the assay incubation steps.

Proximity IHC Experimental—E-Cadherin:Beta-Catenin Positive Proximity

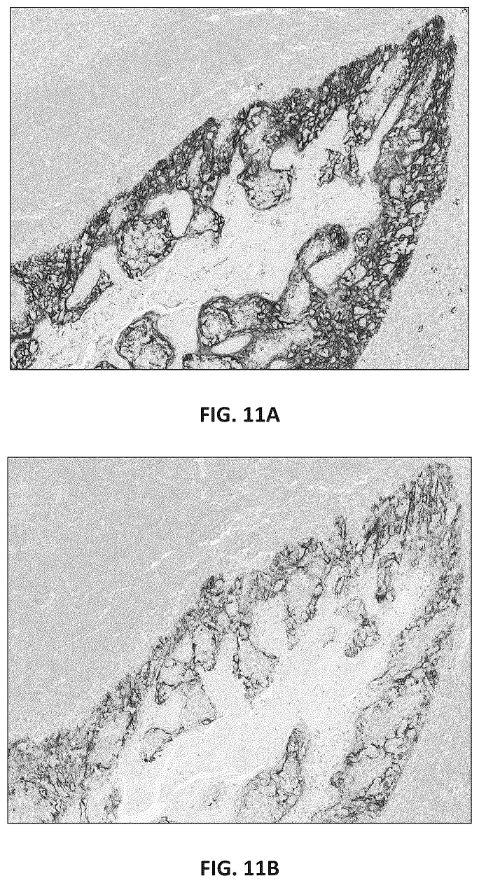

FFPE tonsil tissue was deparaffinized and antigen retrieved (CC1, 60 minutes). Rabbit-anti-E-Cadherin (RTD, 760-4440) and mouse-anti-Beta-catenin (RTD, 760-4242) were co-incubated (about 37° C., about 32 minutes). After washing, a goat polyclonal anti-mouse antibody conjugated to alkaline phosphatase was applied (about 37° C.; about 12 minutes). Following washing, the sample was incubated with a goat polyclonal anti-rabbit antibody conjugated to multiple caged NPs (FIG. 11A) or multiple caged digoxigenins (FIG. 11B) (about 37° C.; about 12 minutes). After washing, the sample was incubated with a mouse-anti-DIG HRP conjugate about (37° C.; about 12 minutes). Tyramide amplification was performed with an Amp HQ kit (RTD, 760-052, about 37° C., about 8 minutes), followed by incubation with a mouse-anti-HQ HRP conjugate (RTD, #760-4602, about 37° C., about 8 minutes). The signal was visualized with DAB and the tissue sections were then counterstained. The slides were dehydrated through a graded ethanol series, cleared with xylene, and coverslipped. The results are shown in FIG. 11A representing the positive proximity signal for E-Cadherin & Beta-Catenin detected using caged NP and FIG. 11B representing the positive proximity signal for E-Cadherin & Beta-Catenin detected using caged DIG.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

Although the disclosure herein has been described with reference to particular embodiments, it is to be understood that these embodiments are merely illustrative of the principles and applications of the present disclosure. It is therefore understood that numerous modifications may be made to the illustrative embodiments and that other arrangements may be devised without departing from the spirit and scope of the present disclosure as defined by the appended claims.

### Additional Embodiments

Additional Embodiment 1. A method of analyzing a sample to determine whether a first target is proximal to a second target, the method comprising:
(a) contacting the sample with an unmasking enzyme-antibody conjugate to form a target-unmasking enzyme-antibody conjugate complex;
(b) contacting the sample with the caged hapten-antibody conjugate to form a target-caged hapten-antibody conjugate complex, wherein the caged hapten-antibody conjugate has any one of Formulas (IVA) and (IVB):

[Specific Binding Entity]-W¹—W²—R¹—O-[DIG]-[Phosphoryl] (IVA),

[Specific Binding Entity]-W¹—W²—R¹—O-[DIG]-PO₄H₂ (IVB),
wherein
W¹ is a bond, or a group comprising a branched or unbranched, substituted or unsubstituted, saturated or unsaturated aliphatic group having between 1 and 10 carbon atoms, and optionally including one or more heteroatoms selected from the group consisting of O, N, or S;
W² is derived from a reactive functional group;
[DIG] is digoxigenin;
[Phosphoryl] is represented by the formula:

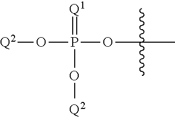

Q¹ is O or S;
Q² is H, —CH₃, or —CH₂CH₃; and
[Specific Binding Entity] is an antibody;
where the group [Phosphoryl] or the group —PO₄H₂ may be attached to any position of [DIG];
(c) unmasking the caged hapten of the target-caged hapten-antibody conjugate complex to form a target-unmasked hapten-antibody conjugate complex;
(d) contacting the sample with first detection reagents to label the first target-unmasked hapten-antibody conjugate complex or the first target; and
(e) detecting the labeled first target-unmasked hapten-antibody conjugate complex or labeled first target.

Additional Embodiment 2. The method of Additional Embodiment 1, wherein Q¹ is O, and at least one Q² is H.

Additional Embodiment 3. The method of Additional Embodiment 2, wherein W² is derived from an amine-reactive group, a thiol-reactive group, and a carbonyl-reactive group.

Additional Embodiment 4. The method of Additional Embodiment 2, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group.

Additional Embodiment 5. The method of Additional Embodiment 1, wherein both Q² groups are H.

Additional Embodiment 6. The method of Additional Embodiment 5, wherein W² is derived from an amine-reactive group, a thiol-reactive group, and a carbonyl-reactive group.

Additional Embodiment 7. The method of Additional Embodiment 5, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group.

Additional Embodiment 8. The method of Additional Embodiment 5, wherein R¹ has the structure depicted in Formula (IIIA):

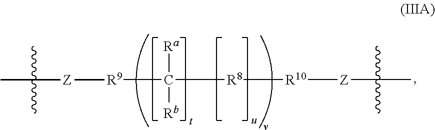

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
   R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Additional Embodiment 9. The method of Additional Embodiment 8, wherein at least one of R^{a} or R^{b} is H.

Additional Embodiment 10. The method of Additional Embodiment 9, wherein R⁸ is O.

Additional Embodiment 11. The method of Additional Embodiment 9, wherein R⁸ is a bond.

Additional Embodiment 12. The method of Additional Embodiment 11, wherein at least one of R^{a} or R^{b} is H.

Additional Embodiment 13. The method of Additional Embodiment 11, wherein both R^{a} and R^{b} are H.

Additional Embodiment 14. The method of Additional Embodiment 12, wherein Z is a bond or —CH₂—.

Additional Embodiment 15. The method of Additional Embodiment 1, wherein R¹ has the structure depicted in Formula (IIIC):

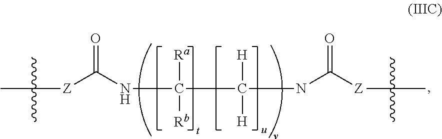

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

Additional Embodiment 16. The method of Additional Embodiment 15, wherein at least one of R^{a} or R^{b} is H.

Additional Embodiment 17. The method of Additional Embodiment 15, wherein Z is a bond or —CH₂—.

Additional Embodiment 18. The method of Additional Embodiment 15, wherein both Q² groups are H.

Additional Embodiment 19. The method of Additional Embodiment 18, wherein W² is derived from an amine-reactive group, a thiol-reactive group, and a carbonyl-reactive group.

Additional Embodiment 20. The method of Additional Embodiment 15, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

Additional Embodiment 21. The method of Additional Embodiment 15, wherein Q¹ is O.

### Further Embodiments

Further Embodiment 1. A caged hapten having any one of Formulas (IA) and (IB):

R²—R¹—O-[DIG]-[Phosphoryl] (IA),

R²—R¹—O-[DIG]-PO₄H₂ (IB),
wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
[DIG] is digoxigenin;
[Phosphoryl] is represented by the formula:

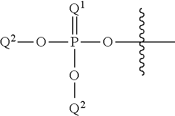

Q¹ is O or S; and
Q² is H, —CH₃, or —CH₂CH₃;
where the group [Phosphoryl] or the group —PO₄H₂ may be attached to any position of [DIG].

Further Embodiment 2. The caged hapten of further embodiment 1, wherein Q¹ is S.

Further Embodiment 3. The caged hapten of further embodiment 1, wherein Q¹ is O, and at least one Q² is H.

Further Embodiment 4. The caged hapten of further embodiment 3, wherein R² is selected from an amine-reactive group, a thiol-reactive group, and a carbonyl-reactive group.

Further Embodiment 5. The caged hapten of further embodiment 3, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

Further Embodiment 6. The caged hapten of further embodiment 1, wherein both Q² groups are H.

Further Embodiment 7. The caged hapten of further embodiment 6, wherein R² is selected from an amine-reactive group, a thiol-reactive group, and a carbonyl-reactive group.

Further Embodiment 8. The caged hapten of further embodiment 6, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

Further Embodiment 9. The caged hapten of further embodiment 6, wherein R¹ has the structure depicted in Formula (IIIA):

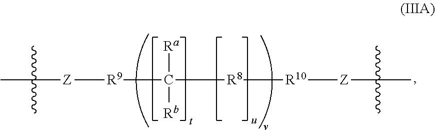

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 10. The caged hapten of further embodiment 9, wherein at least one of R^{a} or R^{b} is H.

Further Embodiment 11. The caged hapten of further embodiment 9, wherein R⁸ is O.

Further Embodiment 12. The caged hapten of further embodiment 9, wherein R⁸ is a bond.

Further Embodiment 13. The caged hapten of further embodiment 12, wherein at least one of R^{a} or R^{b} is H.

Further Embodiment 14. The caged hapten of further embodiment 12, wherein both R^{a} and R^{b} are H.

Further Embodiment 15. The caged hapten of further embodiment 14, wherein Z is a bond or —CH₂—.

Further Embodiment 16. The caged hapten of further embodiment 1, wherein R¹ has the structure depicted in Formula (IIC):

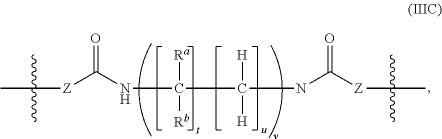

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

Further Embodiment 17. The caged hapten of further embodiment 16, wherein at least one of R^{a} or R^{b} is H.

Further Embodiment 18. The caged hapten of further embodiment 16, wherein Z is a bond or —CH₂—.

Further Embodiment 19. The caged hapten of further embodiment 16, wherein both Q² groups are H.

Further Embodiment 20. The caged hapten of further embodiment 19, wherein R² is selected from an amine-reactive group, a thiol-reactive group, and a carbonyl-reactive group.

Further Embodiment 21. The caged hapten of further embodiment 19, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

Further Embodiment 22. The caged hapten of further embodiment 19, wherein Q¹ is O.

Further Embodiment 23. A caged hapten having Formula (IIID):

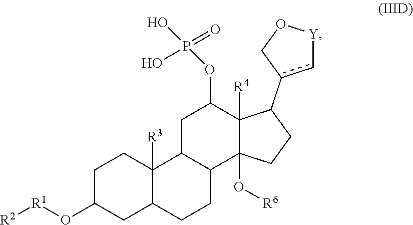

wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
R⁶ is H or a linear or branched or substituted or unsubstituted C₁-C₆ alkyl group;
m, n, and o are each independently 0 or an integer ranging from 1 to 4; and
Y is —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

Further Embodiment 24. The caged hapten of further embodiment 23, wherein R¹ has the structure depicted in Formula (IIIC):

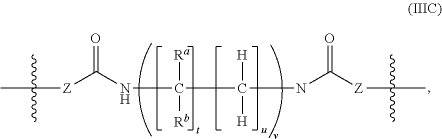

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

Further Embodiment 25. The caged hapten of further embodiment 24, wherein at least one of R^{a} or R^{b} is H.

Further Embodiment 26. The caged hapten of further embodiment 24, wherein Z is a bond or —CH₂—.

Further Embodiment 27. The caged hapten of further embodiment 24, wherein R² is selected from an amine-reactive group, a thiol-reactive group, and a carbonyl-reactive group.

Further Embodiment 28. The caged hapten of further embodiment 24, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

Further Embodiment 29. The caged hapten of further embodiment 24, wherein R² is selected from an amine-reactive group, a thiol-reactive group, and a carbonyl-reactive group.

Further Embodiment 30. The caged hapten of further embodiment 24, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

Further Embodiment 31. The caged hapten of further embodiment 29, wherein R¹ has the structure depicted in Formula (IIIA):

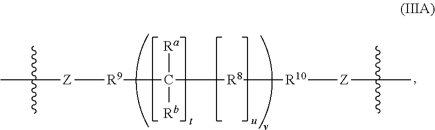

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 32. The caged hapten of further embodiment 31, wherein at least one of R^{a} or R^{b} is H.

Further Embodiment 33. The caged hapten of further embodiment 31, wherein R⁸ is O.

Further Embodiment 34. The caged hapten of further embodiment 31, wherein R⁸ is a bond.

Further Embodiment 35. The caged hapten of further embodiment 34, wherein at least one of R^{a} or R^{b} is H.

Further Embodiment 36. The caged hapten of further embodiment 34, wherein both R^{a} and R^{b} are H.

Further Embodiment 37. The caged hapten of further embodiment 36, wherein Z is a bond or —CH₂—.

Further Embodiment 38. The caged hapten of further embodiment 23, wherein at least one of R³, R⁴, or R⁶ is —CH₃.

Further Embodiment 39. The caged hapten of further embodiment 23, wherein at least one of R³ and R⁴ is —CH₃.

Further Embodiment 40. The caged hapten of further embodiment 39, wherein R⁶ is H.

Further Embodiment 41. The caged hapten of further embodiment 23, wherein R² is H.

Further Embodiment 42. The caged hapten of further embodiment 23, wherein Y is —C(O)—.

Further Embodiment 43. The caged hapten of further embodiment 23, wherein R² is H and Y is —C(O)—.

Further Embodiment 44. The caged hapten of further embodiment 43, wherein R¹ has the structure depicted in Formula (IIIA):

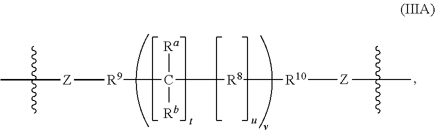

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 45. A conjugate comprising the caged hapten of any one of further embodiments 1-44 and a primary antibody.

Further Embodiment 46. The conjugate of further embodiment 45, wherein the caged hapten is indirectly coupled to the primary antibody.

Further Embodiment 47. The conjugate of further embodiment 46, wherein the primary antibody is an intact primary antibody.

Further Embodiment 48. A conjugate comprising the caged hapten of any one of further embodiments 1-44 and a secondary antibody.

Further Embodiment 49. The conjugate of further embodiment 48, wherein the caged hapten is indirectly coupled to the secondary antibody.

Further Embodiment 50. The conjugate of further embodiment 48, wherein the secondary antibody is an intact secondary antibody.

Further Embodiment 51. A conjugate having any one of Formulas (IVA) and (IVB):

[Specific Binding Entity]-W¹—W²—R¹—O-[DIG]-[Phosphoryl] (IVA),

[Specific Binding Entity]-W¹—W²—R¹—O-[DIG]-PO₄H₂ (IVB),
wherein
W¹ is a bond, or a group comprising a branched or unbranched, substituted or unsubstituted, saturated or unsaturated aliphatic group having between 1 and 10 carbon atoms, and optionally including one or more heteroatoms selected from the group consisting of O, N, or S;
W² is derived from a reactive functional group;
[DIG] is digoxigenin;
[Phosphoryl] is represented by the formula:

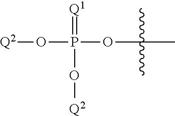

Q¹ is O or S;
Q² is H, —CH₃, or —CH₂CH₃; and
[Specific Binding Entity] is a specific binding entity;
where the group [Phosphoryl] or the group —PO₄H₂ may be attached to any position of [DIG].

Further Embodiment 52. The conjugate of further embodiment 51, wherein Q¹ is O, and at least one Q² is H.

Further Embodiment 53. The conjugate of further embodiment 52, wherein W² is derived from an amine-reactive group, a thiol-reactive group, and a carbonyl-reactive group.

Further Embodiment 54. The conjugate of further embodiment 52, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group.

Further Embodiment 55. The conjugate of further embodiment 51, wherein both Q² groups are H.

Further Embodiment 56. The conjugate of further embodiment 55, wherein W² is derived from an amine-reactive group, a thiol-reactive group, and a carbonyl-reactive group.

Further Embodiment 57. The conjugate of further embodiment 55, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, or an amino group.

Further Embodiment 58. The conjugate of further embodiment 55, wherein R¹ has the structure depicted in Formula (IIIA):

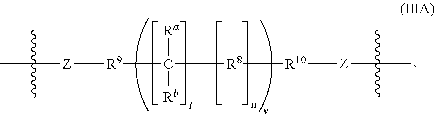

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 59. The conjugate of further embodiment 58, wherein at least one of R^{a} or R^{b} is H.

Further Embodiment 60. The conjugate of further embodiment 59, wherein R⁸ is O.

Further Embodiment 61. The conjugate of further embodiment 59, wherein R⁸ is a bond.

Further Embodiment 62. The conjugate of further embodiment 61, wherein at least one of R^{a} or R^{b} is H.

Further Embodiment 63. The conjugate of further embodiment 61, wherein both R^{a} and R^{b} are H.

Further Embodiment 64. The conjugate of further embodiment 62, wherein Z is a bond or —CH₂—.

Further Embodiment 65. The conjugate of further embodiment 51, wherein R¹ has the structure depicted in Formula (IIIC):

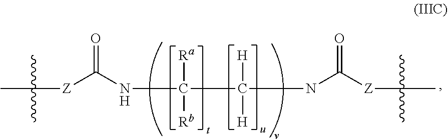

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

Further Embodiment 66. The conjugate of further embodiment 65, wherein at least one of R^{a} or R^{b} is H.

Further Embodiment 67. The conjugate of further embodiment 65, wherein Z is a bond or —CH₂—.

Further Embodiment 68. The conjugate of further embodiment 65, wherein both Q² groups are H.

Further Embodiment 69. The conjugate of further embodiment 68, wherein W² is derived from an amine-reactive group, a thiol-reactive group, and a carbonyl-reactive group.

Further Embodiment 70. The conjugate of further embodiment 68, wherein W² is derived from a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

Further Embodiment 71. The conjugate of further embodiment 65, wherein Q¹ is O.

Further Embodiment 72. A method of analyzing a sample to determine whether a first target is proximal to a second target, the method comprising:
(f) contacting the sample with an unmasking enzyme-antibody conjugate to form a target-unmasking enzyme-antibody conjugate complex;
(g) contacting the sample with the caged hapten-antibody conjugate of any one of further embodiments 45-50 to form a target-caged hapten-antibody conjugate complex;
(h) unmasking the caged hapten of the target-caged hapten-antibody conjugate complex to form a target-unmasked hapten-antibody conjugate complex;
(i) contacting the sample with first detection reagents to label the first target-unmasked hapten-antibody conjugate complex or the first target; and
(j) detecting the labeled first target-unmasked hapten-antibody conjugate complex or labeled first target.

Further Embodiment 73. The method of further embodiment 72, wherein the first detection reagents comprise (i) a secondary antibody specific to the unmasked hapten of the target-unmasked hapten-antibody complex, the secondary antibody conjugated to a first enzyme such that the secondary antibody labels the target-unmasked hapten-antibody complex with the first enzyme; and (ii) a first substrate for the first enzyme.

Further Embodiment 74. The method of further embodiment 73, wherein the first substrate is a chromogenic substrate or a fluorescent substrate.

Further Embodiment 75. The method of further embodiment 72, wherein the first detection reagents include amplification components to label the unmasked enzyme of the target-unmasked hapten-antibody conjugate complex with a plurality of first reporter moieties.

Further Embodiment 76. The method of further embodiment 75, wherein the plurality of first reporter moieties are haptens.

Further Embodiment 77. The method of further embodiment 76, wherein the first detection reagents further comprise secondary antibodies specific to the plurality of first reporter moieties, each secondary antibody conjugated to a second reporter moiety.

Further Embodiment 78. A method for analyzing a sample to determine whether a first target is proximal to a second target, the method comprising:
(a) contacting the sample with an unmasking enzyme-antibody conjugate to form a target-unmasking enzyme-antibody conjugate complex;
(b) contacting the sample with the caged hapten-antibody conjugate of any one of further embodiments 45-50 to form a target-caged hapten-antibody conjugate complex;
(c) unmasking the caged hapten of the target-caged hapten-antibody conjugate complex to form a target-unmasked hapten-antibody conjugate complex;
(d) performing a signal amplification step to label the target-unmasked hapten-antibody conjugate complex with a plurality of reporter moieties; and
(e) detecting the plurality of reporter moieties.

Further Embodiment 79. The method of further embodiment 78, wherein the plurality of reporter moieties are haptens; and wherein the method further comprises introducing secondary antibodies specific to the plurality of first reporter moieties, each secondary antibody conjugated to a second reporter moiety.

Further Embodiment 80. The method of further embodiment 79, wherein the second reporter moiety is an amplification enzyme and wherein the method further comprises introducing a chromogenic substrate or a fluorescent substrate for the amplification enzyme.

Further Embodiment 81. The method of further embodiment 78, further comprising detecting a total amount of target in the sample.

Further Embodiment 82. A method for analyzing a sample to determine whether a first target is proximal to a second target, the method comprising:
(a) contacting the sample with a first detection probe, the first detection probe comprising one of the caged hapten-antibody conjugates of any one of further embodiments 45-50 or an unmasking enzyme-antibody conjugate;
(b) contacting the sample with a second detection probe, the second detection probe comprising the other of the caged hapten antibody conjugate of any one of further embodiments 45-50 or the unmasking enzyme-antibody conjugate;
(c) contacting the sample with at least first detection reagents to label a formed unmasked hapten-antibody conjugate target complex; and
(d) detecting signals from the labeled unmasked hapten-antibody conjugate target complex.

Further Embodiment 83. The method of further embodiment 82, further comprising the step of detecting a total amount of target within the sample.

Further Embodiment 84. The method of further embodiment 82, wherein the first detection reagents include amplification components to label the unmasking enzyme of the first target-unmasked hapten-antibody conjugate complex with a plurality of first reporter moieties.

Further Embodiment 85. The method of further embodiment 83, wherein the plurality of first reporter moieties are haptens.

Further Embodiment 86. The method of further embodiment 84, wherein the first detection reagents further comprise secondary antibodies specific to the plurality of first reporter moieties, each secondary antibody conjugated to a second reporter moiety.

Further Embodiment 87. The method of further embodiment 85, wherein the second reporter moiety is selected from the group consisting of an amplification enzyme or a fluorophore.

Further Embodiment 88. The method of further embodiment 85, wherein the second reporter moiety is an amplification enzyme and wherein the first detection reagents further comprise a first chromogenic substrate or fluorescent substrate for the amplification enzyme.

Further Embodiment 89. The method of further embodiment 82, wherein the method further comprises a decaging step.

Further Embodiment 90. A caged hapten having Formula (IIIA):

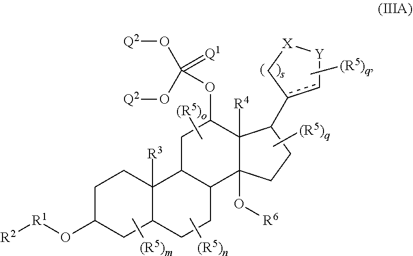

wherein
Q¹ is O or S;
Q² is H, —CH₃, or —CH₂CH₃;
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
each R⁵ is independently H, —CH₃, —CH₂CH₃, a halogen, or —C(O)H;
R⁶ is H or a linear or branched or substituted or unsubstituted C₁-C₆ alkyl group;
m, n, and o are each independently 0 or an integer ranging from 1 to 4;
p and q are each independently 0 or an integer ranging from 1 to 3;
s is 1 or 2; and
X and Y are each independently —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

Further Embodiment 91. The caged hapten of further embodiment 90, wherein R¹ has the structure depicted in Formula (IIIC):

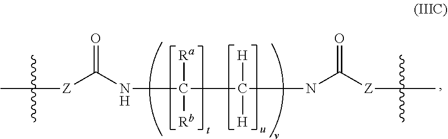

wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

Further Embodiment 92. The caged hapten of further embodiment 91, wherein at least one of R^{a} or R^{b} is H.

Further Embodiment 93. The caged hapten of further embodiment 91, wherein Z is a bond or —CH₂—.

Further Embodiment 94. The caged hapten of further embodiment 93, wherein R² is selected from an amine-reactive group, a thiol-reactive group, and a carbonyl-reactive group.

Further Embodiment 95. The caged hapten of further embodiment 93, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

Further Embodiment 96. The caged hapten of further embodiment 91, wherein R² is selected from an amine-reactive group, a thiol-reactive group, and a carbonyl-reactive group.

Further Embodiment 97. The caged hapten of further embodiment 91, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, an amino group.

Further Embodiment 98. The caged hapten of further embodiment 97, wherein R¹ has the structure depicted in Formula (IIIA):

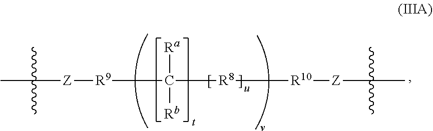

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 99. The caged hapten of further embodiment 98, wherein at least one of R^{a} or R^{b} is H.

Further Embodiment 100. The caged hapten of further embodiment 98, wherein R⁸ is O.

Further Embodiment 101. The caged hapten of further embodiment 98, wherein R⁸ is a bond.

Further Embodiment 102. The caged hapten of further embodiment 101, wherein at least one of R^{a} or R^{b} is H.

Further Embodiment 103. The caged hapten of further embodiment 101, wherein both R^{a} and R^{b} are H.

Further Embodiment 104. The caged hapten of further embodiment 103, wherein Z is a bond or —CH₂—.

Further Embodiment 105. The caged hapten of further embodiment 90, wherein at least one of R³, R⁴, or R⁶ is —CH₃.

Further Embodiment 106. The caged hapten of further embodiment 90, wherein at least one of R³ and R⁴ is —CH₃.

Further Embodiment 107. The caged hapten of further embodiment 106, wherein R⁶ is H.

Further Embodiment 108. The caged hapten of further embodiment 90, wherein R² is H.

Further Embodiment 109. The caged hapten of further embodiment 90, wherein Y is —C(O)—.

Further Embodiment 110. The caged hapten of further embodiment 90, wherein R² is H and Y is —C(O)—.

Further Embodiment 111. The caged hapten of further embodiment 110, wherein R¹ has the structure depicted in Formula (IIIA):

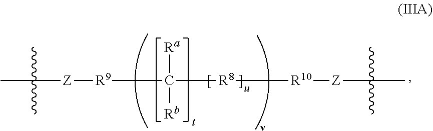

wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c}(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

Further Embodiment 112. The caged hapten of further embodiment 90, wherein the caged hapten has Formula (IIB):

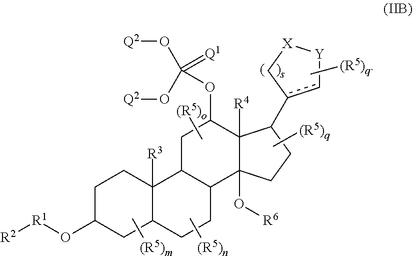

Further Embodiment 113. The caged hapten of further embodiment 90, wherein the caged hapten has Formula (IIIC):

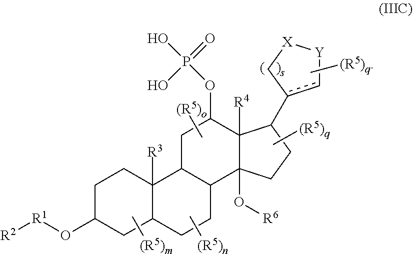

Further Embodiment 114. The caged hapten of further embodiment 90, wherein the caged hapten has Formula (IIIE):

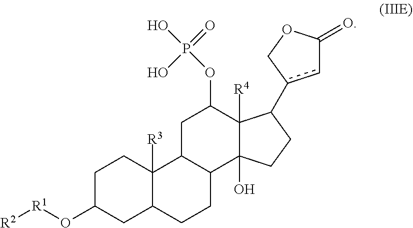

Further Embodiment 115. The caged hapten of further embodiment 90, wherein the caged hapten has Formula (IIIF):

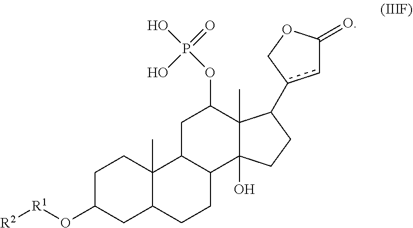

Further Embodiment 116. A conjugate comprising the caged hapten of any one of further embodiments 90-115 and a primary antibody.

Further Embodiment 117. The conjugate of further embodiment 116, wherein the caged hapten is indirectly coupled to the primary antibody.

Further Embodiment 118. The conjugate of further embodiment 117, wherein the primary antibody is an intact primary antibody.

Further Embodiment 119. A method of analyzing a sample to determine whether a first target is proximal to a second target, the method comprising:
(a) contacting the sample with an unmasking enzyme-antibody conjugate to form a target-unmasking enzyme-antibody conjugate complex;
(b) contacting the sample with the caged hapten conjugate of any one of further embodiments 116-118 to form a target-caged hapten-antibody conjugate complex;
(c) unmasking the caged hapten of the target-caged hapten-antibody conjugate complex to form a target-unmasked hapten-antibody conjugate complex;
(d) contacting the sample with first detection reagents to label the first target-unmasked hapten-antibody conjugate complex or the first target; and
(e) detecting the labeled first target-unmasked hapten-antibody conjugate complex or labeled first target.

Further Embodiment 120. The method of further embodiment 119, wherein the first detection reagents comprise (i) a secondary antibody specific to the unmasked hapten of the target-unmasked hapten-antibody complex, the secondary antibody conjugated to a first enzyme such that the secondary antibody labels the target-unmasked hapten-antibody complex with the first enzyme; and (ii) a first substrate for the first enzyme.

1. A caged hapten having any one of Formulas (IA) and (IB):
R²—R¹—O-[DIG]-[Phosphoryl] (IA),
R²—R¹—O-[DIG]-PO₄H₂ (IB),
wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
[DIG] is digoxigenin;
[Phosphoryl] is represented by the formula: 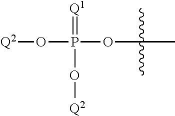
Q¹ is O or S; and
Q² is H, —CH₃, or —CH₂CH₃;
where the group [Phosphoryl] or the group —PO₄H₂ may be attached to any position of [DIG].

2. The caged hapten of claim 1, wherein Q¹ is S.

3. The caged hapten of claim 1, wherein Q¹ is O, and at least one Q² is H.

4. The caged hapten of claim 3, wherein R² is selected from the group consisting of a dibenzocyclooctyne, a trans-cyclooctene, an alkyne, an alkene, an azide, a tetrazine, a maleimide, a N-hydroxysuccinimide, a thiol, a 1,3-nitrone, an aldehyde, a ketone, a hydrazine, a hydroxylamine, and an amino group.

5. The caged hapten of claim 1, wherein both Q² groups are H.

6. The caged hapten of claim 5, wherein R¹ has the structure depicted in Formula (IIIA): 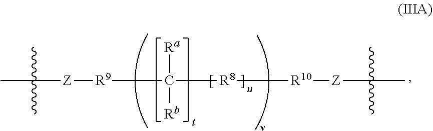
wherein
R⁸ is a bond, —O—, —S—, —C(R^{c})(R^{d})—, or —N(R^{c})—;
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
t and u are each independently 0, 1, or 2, provided that t+u is at least 1; and
v is an integer ranging from 1 to 8.

7. The caged hapten of claim 6, wherein at least one of R^{a} or R^{b} is H.

8. The caged hapten of claim 6, wherein R⁸ is O.

9. The caged hapten of claim 6, wherein R⁸ is a bond.

10. The caged hapten of claim 9, wherein at least one of R^{a} or R^{b} is H.

11. The caged hapten of claim 9, wherein both R^{a} and R^{b} are H.

12. The caged hapten of claim 11, wherein Z is a bond or —CH₂—.

13. The caged hapten of claim 1, wherein R¹ has the structure depicted in Formula (IIIC): 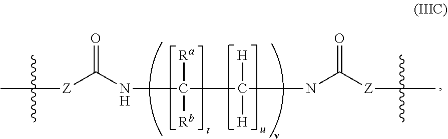
wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

14. A caged hapten having Formula (IIID): 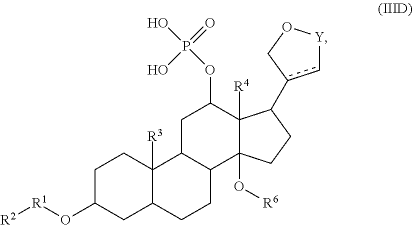
wherein
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
R⁶ is H or a linear or branched or substituted or unsubstituted C₁-C₆ alkyl group;
m, n, and o are each independently 0 or an integer ranging from 1 to 4; and
Y is —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

15. The caged hapten of claim 14, wherein R¹ has the structure depicted 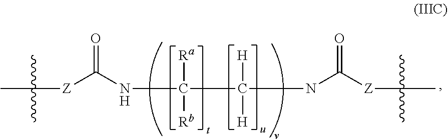
wherein
R^{a} and R^{b} are each independently H, a C₁-C₄ alkyl group, F, Cl, or —N(R^{c})(R^{d});
R^{c} and R^{d} are each independently selected from H or —CH₃;
R⁹ and R¹⁰ are each independently a bond or a group selected from carbonyl, amide, imide, ester, ether, amine, thione, thiol;
each Z is independently a bond, —CH₂—, —CH₂CH₂—, or —CH₂CH₂CH₂—;
u and t are each independently 0, 1, or 2, provided that u+t is at least 1; and
v is an integer ranging from 1 to 8.

16. The caged hapten of claim 15, wherein Z is a bond or —CH₂—.

17. A conjugate comprising the caged hapten of claim 1, and a primary antibody.

18. A conjugate comprising the caged hapten of claim 1, and a secondary antibody.

19. A caged hapten having Formula (IIIA): 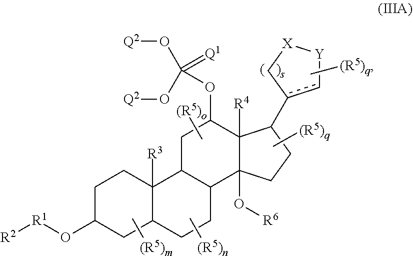
wherein
Q¹ is O or S;
Q² is H, —CH₃, or —CH₂CH₃;
R¹ is a bond, or a group comprising a branched or unbranched, substituted or unsubstituted, saturated or unsaturated aliphatic group having between 1 and 30 carbon atoms, and optionally including one or more heteroatoms selected from the group consisting of O, N, or S;
R² is H or a reactive functional group;
R³ is H, —CH₃, —CH₂CH₃, —OH, or —O-Me;
R⁴ is H, —CH₃, or —CH₂CH₃, —OH, or —O-Me;
each R⁵ is independently H, —CH₃, —CH₂CH₃, a halogen, or —C(O)H;
R⁶ is H or a linear or branched or substituted or unsubstituted C₁-C₆ alkyl group;
m, n, and o are each independently 0 or an integer ranging from 1 to 4;
p and q are each independently 0 or an integer ranging from 1 to 3;
s is 1 or 2; and
X and Y are each independently —CH₂—, —C(R⁷)—, —N(H)—, —N(R⁷)—, —O—, or —S—, or —C(O)—, where R⁷ is a C₁-C₄ linear or branched, substituted or unsubstituted alkyl group.

20. The caged hapten of claim 19, wherein the caged hapten has any one of Formulas (IIB), (IIIC), (IIIE), or (IIIF): [chemical structure]
